(19) United States
(12) Patent Application Publication
(10) Pub. No.: US 2012/0029085 A1
(43) Pub. Date: Feb. 2, 2012

(54) STABILIZATION OF QUINOL COMPOSITION SUCH AS CATECHOLAMINE DRUGS

(76) Inventor: Jon MacKay, Spokane, WA (US)

(21) Appl. No.: 12/846,656

(22) Filed: Jul. 29, 2010

(51) Int. Cl. A61K 31/167; A61P 23/00; A61K 31/138

(52) U.S. Cl. 514/626; 514/653

(57) ABSTRACT

Compositions and methods are provided for obtaining stabilized quinol compositions, such as catecholamine drugs (e.g., epinephrine solutions), and also for obtaining stable pharmaceutical formulations that comprise a stabilized quinol composition and a second pharmacologically active component such as a local anesthetic or other active drug ingredient having a reversibly protonated amine group. Stability is achieved through the inclusion of an appropriately selected pH buffer and a thiol agent, based on redox and pH buffering principles including pKa of the buffer and of the reversibly protonated amine group.

### BACKGROUND OF THE INVENTION

1. Field of the Invention

The present invention relates to compositions and methods for preserving and maintaining the structural integrity, chemical stability and biological activity of quinol-containing compositions. More specifically, the invention relates to improved stability of quinol-containing compositions such as epinephrine and other catecholamine drugs, and of pharmaceutical formulations that include quinol compositions and other active drugs such as drugs having amine groups that can be reversibly protonated.

2. Description of the Related Art

A number of chemical compounds having uses in the drug and food industries for a variety of purposes exhibit instabilities leading to oxidative degradation, which compromises their effectiveness and engenders undesirable costs associated with obtaining fresh reagents, discarding degraded reagents, and monitoring inventories of reagents that have only limited shelf-life. Among such chemical compounds are those that contain a quinol (dihydroxybenzene) moiety which can detrimentally undergo oxidative degradation to a corresponding quinone structure, which in turn may be compromised by further chemical degradation.

Exemplary compounds include members of the catecholamines (e.g., epinephrine, norepinephrine, levonordefrin; see, e.g., U.S. Pat. No. 5,002,973.), a family of compounds which includes naturally occurring neurotransmitters and also includes a number of synthetic products having applications as drugs in a wide variety of indications.

Catecholamines and other quinol compounds are susceptible to oxidation in solution (e.g., aqueous solution) that may be accompanied by a loss of pharmacological activity, and under current storage practices such oxidized compounds can be further converted to degradation products having potentially harmful properties. For instance, the catecholamine epinephrine is rapidly oxidized in aqueous solution, degrading to adrenochrome and adrenalone. (e.g., Kalyanaraman et al., 1984 J. Biol. Chem. 259:354; Kirchhoefer et al., 1986 Am J. Hosp. Pharm. 43:1741; Stepensky et al., 2004 J. Pharm Sci. 93:969; Newton et al., 1981 Am. J. Hosp. Pharm. 38:1314) At acidic pH values, degradation of epinephrine has also been reported to result from conversion of the biologically active L-enantiomer to the inactive D-enantiomer, yielding a racemic mixture of undesirably reduced potency (Stepensky et al., 2004 J. Pharm Sci. 93:969).

Presently available pharmaceutical formulations of catecholamine drug products and their structurally related analogues are typically plagued by efforts to stabilize the catecholamines, which efforts often result in disadvantageous and unwanted properties of the product. Many current epinephrine formulations, for example, contain bisulfite and/or metabisulfite additives that are included as mild reducing agents, and which are believed to inhibit oxidative degradation of the catecholamine. (e.g., Dalton-Bunnow, 1985 Am. J. Hosp. Pharm. 42:2220; Grubstein et al., 1992 Drug Dev. Ind. Pharm. 18:1549) These reducing agents, however, readily react with epinephrine to generate sulfonated derivatives that lack epinephrine biological activity. (e.g., Schroeter et al. 1958 J. Am. Pharmaceut. Assoc. 47:723; Hajratwala, 1975 J. Pharmaceut. Sci. 64:45) Additionally, allergic reactions to the bisulfite preservatives are often observed, such that formulations containing bisulfites will be contraindicated in individuals having such allergies. (e.g., Campbell et al., 2001 Anesth. Prog. 48:21; Smolinske, 1992 Clin. Toxicol. 30:597) Moreover, epinephrine is unstable in solution for even brief time periods and must be kept at an acidic pH in order to avoid extremely rapid degradation that is associated with attempts to prepare epinephrine solutions having neutral pH values. (Robinson et al., 2000 Anesthesia 55:853; Newton et al., 1981 Am. J. Hosp. Pharm. 38:1314)

Another problem associated with efforts to provide storage conditions for quinol compounds relates to pharmaceutical formulations that contain a quinol compound along with a second pharmaceutical agent. For example, the quinol compound epinephrine is often included for its desirable pharmacological activity as a vasoconstrictor in formulations of local anesthetics, including amino ester local anesthetics (e.g., procaine) and amino amide local anesthetics (e.g., lidocaine). Many of these local anesthetics comprise an amine-containing compound having at least one amine group that is capable of being reversibly protonated. Such pharmaceutical formulations are typically provided in relatively acidic condition (e.g., pH<4) in an effort to preserve the quinol compound, which as described above, tends to degrade rapidly at pH values closer to neutrality.

Acidic formulations of such local anesthetics, however, suffer from other drawbacks, in particular, the problem that the low pH favors the presence of the protonated form of the reversibly protonated amine group. This problem manifests itself in an undesirably delayed onset of the desired pharmacological activity—anesthetic effect—insofar as the charge of the protonated amine group hinders the ability of the local anesthetic to traverse cellular membranes for purposes of exerting its pharmacological activity intracellularly. Hence, the anesthetic effect is delayed, and the efficiency of drug utilization at the desired local site is decreased by circulatory system clearance from the region of protonated drug molecules that have not yet equilibrated with the deprotonated form in the extracellular environment as a prelude to plasma membrane transit. Moreover, the acidic pH of such formulations typically results in pain experienced by the recipient at the site of injection, a seemingly inevitable consequence of the low pH used to protect the quinol compound.

Clearly there remains a need for improved formulations of active drug compounds such as quinol compounds, and for improved pharmaceutical formulations containing both quinol compounds and active drug compounds having amine groups that can be reversibly protonated. The present invention provides improved compositions and methods that address these needs, and offers other related advantages.

### BRIEF SUMMARY OF THE INVENTION

In certain embodiments, the present invention provides a stable pharmaceutical formulation, comprising (a) a first composition that comprises at least one quinol compound having a first desired pharmacological activity; (b) a second composition that comprises at least one local anesthetic compound, said local anesthetic compound comprising at least one amine group that is capable of being reversibly protonated, and being capable of reversibly binding to a voltage-gated Na⁺ channel in a cell membrane to thereby alter Na⁺ movement through the voltage-gated Na⁺ channel; (c) at least one thiol agent; and (d) at least one pH buffer that maintains a substantially constant pH in the pharmaceutical formulation, wherein the pH is greater than about pH 5.5. In certain embodiments the quinol compound is present in a reduced form. In certain embodiments the quinol compound comprises an ortho-quinol moiety or a para-quinol moiety. In certain embodiments at least one quinol compound comprises a catecholamine.

In certain embodiments at least one quinol compound comprises a compound of Formula (I):

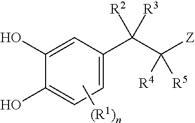

wherein:

n is 0, 1, 2 or 3

each R¹ is the same or different and independently hydrogen, alkyl, hydroxyl, alkoxide, —OC(O)alkyl, —OC(O)aralkyl, aralkyl, amino or halo;

R² and R³ are the same or different and independently hydrogen, hydroxyl, alkoxide, alkyl, oxo, —OC(O)alkyl, —OC(O)aralkyl, amino, monoalkylamino, dialkylamino or halo;

R⁴ and R⁵ are the same or different and independently hydrogen, hydroxyl, alkoxide, —NR⁶₂, —NHNH₂ or lower alkyl,

Z is —NR⁶₂, —COOH or —CR⁷₃;

each R⁶ is the same or different and independently hydrogen, alkyl, aralkyl; or

R⁵ and R⁶ together with the atoms to which they are attached form a heterocycle; and

each R⁷ is the same or different and independently hydrogen, alkyl, aralkyl, —COOH, amino, —C(O)Oalkyl, —C(O)Oaryl, —C(O)Oaralkyl, —NHNH₂, monoalkylamino, dialkylamino,

as a single stereoisomer, a mixture of stereoisomers, or as a racemic mixture of stereoisomers; or as a pharmaceutically acceptable salt thereof.

In certain embodiments the quinol compound comprises a compound selected from 1,2-dihydroxybenzene (catechol, pyrocatechol), 1,4-dihydroxybenzene, epinephrine, norepinephrine, dopamine, dobutamine, isoproterenol, racepinephrine, arbutamine, carbidopa, deoxyepinephrine, dioxethedrine, 3-(3,4-dihydroxyphenyl)-alanine (L-, D- or DL-DOPA), dopexamine, droxidopa, ethylnorepinephrine, hexoprenaline, isoetharine, methyldopa, N-methylepinephrine, nordefrin, rimiterol, epinephrine bitartrate, L-epinephrine-D-hydrogentartate, adrenalone (CAS 99-45-6), arbutamine (CAS 128470-16-6), benserazide (CAS 322-35-0), carbidopa (CAS 28860-95-9), deoxyepinephrine (CAS 501-15-5), dioxethdrine (CAS 497-75-6), dobutamine (CAS 34368-04-02), dopa (CAS 63-84-3), dopamine (CAS 51-61-6), dopexamine (CAS 86197-47-9), droxidopa (CAS 23651-95-8), epinephrine (CAS 51-43-4), ethylnorepinephrine (CAS 536-24-3), fluorodopa (CAS 92812-82-3), hexoprenaline (CAS 3215-70-1), isoetharine (CAS 530-08-5), isoproterenol (CAS 7683-59-2), levodopa (CAS 59-92-7), methyldopa (CAS 555-30-6), N-methylepinephrine (CAS 554-99-4), nordefrin (CAS 6539-57-7), norepinephrine (CAS 51-41-2), protokylol (CAS 136-70-9), rimiterol (CAS 32953-89-2), nordihydroguaiaretic acid and tetrahydropapaveroline (CAS 4747-99-3). In certain embodiments the quinol compound comprises epinephrine.

In certain embodiments the thiol agent is selected from cysteine, N-acetylcysteine, glutathione, monothioglycerol, cysteine ethyl ester, homocysteine, Coenzyme A, dithiothreitol, 2-mercaptoethanol, 2,3-dimercapto-1-propanol, 2,3-butanedithiol, 2-mercaptoethylamine, ethanedithiol, propanedithiol, 3-mercapto-2-butanol, dimercapto-propane-1-sulfonic acid, dimercaptosuccinic acid, trithiocyanuric acid, 2,5-dimercapto-1,3,4-thiadiazole, 3,4-dimercaptotoluene, 1,4-dimercapto-2,3-butanediol, 1,3-propanedithiol, 1,4-butanedithiol, N-Acetylpenicillamine, ACV, N-amyl mercaptan, bucillamine, N-butyl mercaptan, sec-butyl bercaptan, tert-butyl mercaptan, captopril, cysteamine, DBHBT, 2,3-dimercapto-1-propanesulfonic acid, dimercaprol, dithiosalicylic acid, 1,2-ethanedithiol, ethanethiol, isobutyl mercaptan, mecysteine, 2-mercaptoethanol, MESNA, methanethiol, pantetheine, penicillamine, 1,3-propanedithiol, succimer, thioacetic acid, thiobenzyl alcohol, thiocyanic acid, thioglycerol, thioglycolic acid, thiolactic acid, thiomalic acid, thionalide, 1-thiosorbitol, tiopronin, tixocortol and trithiocarbonic acid. In certain embodiments the thiol agent is N-acetylcysteine.

In certain embodiments the pH buffer is present under conditions and in sufficient quantity to maintain a pH that is from about pH 5.5 to about pH 9.0, or from about pH 5.5 to about pH 8.5, or from about pH 5.5 to about pH 8.25, or from about pH 5.75 to about pH 7.75, or from about pH 6.0 to about pH 7.5, or from about pH 6.6 to about pH 7.3, or from about pH 6.5 to about pH 7.1, or from about pH 6.3 to about pH 6.9. In certain embodiments the pH buffer comprises a compound that is selected from Tris (8.3), Tricine (8.15), citrate (pKa₃=5.4), acetate (4.75), phosphate (7.2), borate (9.24), HEPES (7.55), HEPPS (8), MES (6.15), ACES (6.9), imidazole (7), diethylmalonic acid (7.2), MOPS (7.2), PIPES (6.8), TES (7.5), carbonate, bicarbonate, malate, pyridine, piperazine, succinate, histidine, maleate, Bis-Tris, pyrophosphate, histidine, MOPSO, BES, DIPSO, MOBS, TAPSO, triethanolamine, POPSO, cacodylic acid, ADA, Bis-Tris propane and HEPPSO. In certain embodiments the pH buffer comprises sodium phosphate. In certain embodiments the quinol compound comprises epinephrine, the thiol agent is N-acetylcysteine and the pH buffer comprises sodium phosphate.

In certain embodiments the amine group that is capable of being reversibly protonated has a pKa of from about pH 7.5 to about pH 9.3, or a pKa of from about pH 7.6 to about pH 9.2, or a pKa of from about pH 7.7 to about pH 9.1, or a pKa of from about pH 7.8 to about pH 9.0, or a pKa of from about pH 7.9 to about pH 8.9, or a pKa of from about pH 8.0 to about pH 8.8, or a pKa of from about pH 8.1 to about pH 8.7, or a pKa of from about pH 8.2 to about pH 8.6, or a pKa of from about pH 8.3 to about pH 8.5.

In certain embodiments the local anesthetic compound is selected from the group consisting of an amino ester anesthetic and an amino amide anesthetic. In certain embodiments the cell membrane is a plasma membrane. In certain embodiments the cell membrane is present in a neuron. In certain embodiments the cell membrane is selected from a plasma membrane, a mitochondrial membrane, an endoplasmic reticulum membrane, a lysozomal membrane, an exocytic vacuolar membrane and an endocytic vacuolar membrane. In certain embodiments the second composition comprises a compound that is selected from lidocaine, propoxycaine, procaine, prilocalne, bupivacaine, Articaine, Benzocaine, Chloroprocaine, Cocaine, Dibucaine, Etidocaine, Hexylcaine, Mepivicaine, Piperocaine, Ropivacaine and Tetracaine. In certain embodiments the first composition comprises epinephrine and the second composition comprises lidocaine. In certain embodiments the first composition comprises epinephrine, the second composition comprises lidocaine, and the thiol agent comprises N-acetylcysteine. In certain embodiments the first composition comprises epinephrine, the second composition comprises lidocaine, the thiol agent comprises N-acetylcysteine and the pH buffer comprises sodium phosphate. In certain embodiments the stable pharmaceutical formulation comprises a first composition that comprises at least one quinol compound having a first desired pharmacological activity; a second composition that comprises at least one local anesthetic compound that is capable of reversibly binding to a voltage-gated Na⁺ channel in a cell membrane to thereby alter Na⁺ movement through the voltage-gated Na⁺ channel; at least one thiol agent; and at least one pH buffer that maintains a substantially constant pH in the pharmaceutical formulation, wherein the pH is greater than about pH 5.5. In one further embodiment the local anesthetic compound is lidocaine. In certain other further embodiments of the above-described stable pharmaceutical formulations, the formulation comprises an acceptable pharmaceutical carrier.

In certain other embodiments of the above described stable pharmaceutical formulations, the first composition is present at a mass-to-volume ratio in the formulation that is between 1:500,000 and 1:10,000. In certain other further embodiments, the isolated quinol compound is present at one of: (a) at least from about 0.05 percent weight-to-volume to about 0.5 percent weight-to-volume, (b) at least from about 0.01 percent weight-to-volume to about one percent weight-to-volume, (c) at least from about 0.5 percent weight-to-volume to about two percent weight-to-volume, (d) at least from about 0.75 percent weight-to-volume to about three percent weight-to-volume, (e) at least from about one percent weight-to-volume to about four percent weight-to-volume, (f) at least from about two percent weight-to-volume to about five percent weight-to-volume, and (g) at least from about 2.5 percent weight-to-volume to about six percent weight-to-volume.

Turning to other embodiments, the present invention provides a method of stabilizing a quinol compound, comprising contacting (a) at least one isolated quinol compound; (b) at least one thiol agent; and (c) a pH buffer that maintains a substantially constant pH, and thereby stabilizing the quinol compound. In another embodiment there is provided a method of stabilizing a quinol compound, comprising contacting (a) at least one isolated quinol compound; (b) at least one thiol agent; and (c) a pH buffer that maintains a substantially constant pH, to produce a stabilized quinol composition, wherein said stabilized quinol composition comprises the stabilized quinol composition as described above, and thereby stabilizing the quinol compound. In another embodiment there is provided a method of stabilizing a pharmaceutical formulation, comprising contacting (a) a pharmaceutical formulation, (b) at least one thiol agent, and (c) a pH buffer that maintains a substantially constant pH, wherein the pH is greater than about pH 5.5, wherein the pharmaceutical formulation of (a) comprises (i) a first composition that comprises at least one quinol compound having a first desired pharmacological activity, and (ii) a second composition that comprises at least one local anesthetic compound, said local anesthetic compound comprising an amine-containing compound having a second desired pharmacological activity and at least one amine group that is capable of being reversibly protonated, and thereby stabilizing the pharmaceutical formulation.

In another embodiment there is provided a method of stabilizing a pharmaceutical formulation, comprising: contacting (a) a pharmaceutical formulation which comprises (i) a first composition that comprises at least one quinol compound having a first desired pharmacological activity, and (ii) a second composition that comprises at least one local anesthetic compound, said local anesthetic compound comprising an amine-containing compound having a second desired pharmacological activity and at least one amine group that is capable of being reversibly protonated, (b) at least one thiol agent, and (c) a pH buffer that maintains a substantially constant pH, wherein according to certain further embodiments the pH is greater than about pH 5.5, to produce a stable pharmaceutical formulation, wherein said stable pharmaceutical formulation comprises the stable pharmaceutical formulation as described above, and thereby stabilizing the pharmaceutical formulation.

In certain other embodiments the invention provides a method of treating a subject, comprising administering to the subject a stabilized quinol composition, comprising (a) at least one isolated quinol compound; (b) at least one thiol agent; and (c) at least one pH buffer that maintains a substantially constant pH in the stabilized quinol composition, wherein in certain further embodiments the pH is greater than about pH 5.5. In certain further embodiments the stabilized quinol composition comprises at least any one of the above-described stabilized quinol compositions.

In certain other embodiments the invention provides method of treating a subject, comprising administering to said subject a stable pharmaceutical formulation, comprising (a) a first composition that comprises at least one quinol compound having a first desired pharmacological activity; (b) a second composition that comprises at least one amine-containing compound having a second desired pharmacological activity and at least one amine group that is capable of being reversibly protonated; (c) at least one thiol agent; and (d) at least one pH buffer that maintains a substantially constant pH in the pharmaceutical formulation, wherein in certain further embodiments the pH is greater than about pH 5.5. In certain other further embodiments the stable pharmaceutical formulation comprises at least any one of the stable pharmaceutical formulations as described above.

In certain other embodiments the invention provides a method for the manufacture of a medicament for therapeutic treatment of a subject with a stabilized quinol composition, said method comprising contacting (a) at least one isolated quinol compound; (b) at least one thiol agent; and (c) a pH buffer that maintains a substantially constant pH, wherein in certain further embodiments the pH is greater than about pH 5.5. In certain other further embodiments the stabilized quinol composition comprises at least any one of the above described stabilized quinol compositions. In another embodiment there is provided a method for the manufacture of a medicament for therapeutic treatment of a subject with a stable pharmaceutical formulation, said method comprising contacting (a) a pharmaceutical formulation, (b) at least one thiol agent, and (c) a pH buffer that maintains a substantially constant pH, wherein the pH is greater than about pH 5.5, wherein the pharmaceutical formulation of (a) comprises (i) a first composition that comprises at least one quinol compound having a first desired pharmacological activity, and (ii) a second composition that comprises at least one local anesthetic compound, said local anesthetic compound comprising an amine-containing compound having a second desired pharmacological activity and at least one amine group that is capable of being reversibly protonated. In certain further embodiments the stable pharmaceutical formulation comprises at least any one of the above-described stable pharmaceutical formulations.

These and other aspects of the present invention will become apparent upon reference to the following detailed description. All references disclosed herein are hereby incorporated by reference in their entirety as if each was incorporated individually.

### BRIEF DESCRIPTION OF THE DRAWING(S)

FIG. 1 shows a table of the analysis by appearance of quinol containing solutions (epinephrine) with and without a thiol agent (cysteine).

FIG. 2 shows a graph of the relationship between reducing potential (voltage) and pH for cysteine as an exemplary thiol agent.

### DETAILED DESCRIPTION OF THE INVENTION

The present invention relates to compositions and methods whereby quinol compounds such as catecholamines may be beneficially and desirably stabilized in solution. Contrary to long held beliefs that acidic pH environments are needed to stabilize quinol compounds, unexpectedly and as disclosed herein for the first time, such compounds may be protected from oxidative damage at higher pH levels than have been commonly believed to be capable of supporting stable preservation of such compounds, including pH levels at or near the physiological pH values typically found in mammalian tissues (e.g., human plasma, skin, muscle tissue, etc.). In certain preferred embodiments, stabilized quinol compositions may be obtained that display little or no oxidative degradation over extended periods of time, by storage in solution in the presence of thiol-containing compounds and suitable buffers under conditions and in sufficient quantities to maintain a substantially constant pH. Surprisingly, stabilized quinol compositions and related methods as disclosed herein also provide the advantage of resisting degradation by racemization of biologically and/or pharmacologically active enantiomeric forms, thereby preserving the potency of such quinol compounds by inhibiting the appearance of racemic mixtures containing inactive enantiomers.

The invention will in certain embodiments accordingly offer the advantages of a stabilized quinol composition, such as advantages associated with longer shelf-life, convenience of not having to reconstitute a dried or concentrated preparation immediately prior to use, reduced costs for maintaining and replacing inventory, reduced waste of materials and energy caused by having to discard degraded and out-dated preparations and packaging materials, and related economic efficiencies. In certain embodiments the invention will find uses in a variety of contexts such as in pharmacies, hospitals and/or homes, in mobile or emergency medical aid kits, for travelers (especially to remote areas), for medical facilities lacking reliable refrigerated storage or hygienic conditions for sterile reconstitution of an injectable drug, and other contexts where stable, long-term storage of a stable pharmaceutical solution at ambient temperature may offer convenience, safety and/or cost-savings. Certain embodiments thus stabilize quinol compounds by protecting them against oxidative damage in aqueous solution, even during prolonged storage at typical ambient temperatures (e.g., 18, 19, 20, 21, 22, 23, 24, 25, 26 or 27° C.), or at lower or higher temperatures.

The present invention also offers the further unexpected advantage of decreasing the discomfort associated with administration of quinol compositions of the prior art, and of pharmaceutical formulations that contain a quinol composition along with another pharmacologically active ingredient, which compositions and formulations have typically comprised substantially acidic solutions (e.g., pH 2-4) and as such cause pain upon hypodermic injection into a subject. By contrast, according to certain embodiments of the present invention a pH buffer is present that maintains a substantially constant pH in the stabilized quinol composition, preferably a pH that is at or near a physiological pH at the site of administration into the body of the subject. Such a pH may in certain embodiments be from about pH 5.5 to about pH 9, from about pH 5.5 to about pH 8.5, from about pH 5.5 to about pH 8.25, from about pH 5.5 to about pH 8.0, from about pH 5.75 to about pH 7.75, more preferably about pH 6 to about pH 8, from about pH 6 to about pH 7.5, from about pH 6.6 to about pH 7.3, about pH 6.5 to about pH 7.1, about pH 6.3 to about pH 6.9, about pH 6.5 to about pH 7.8, about pH 6.8 to about pH 7.6, about 7 to about 7.5, or from about pH 7.1 to about 7.4, preferably from about pH 7.2 to about 7.4, from about pH 7.3 to about 7.6, or another pH that those familiar with the art will recognize is substantially similar to the pH at a physiological site of parenteral administration of the composition, such that the pain induced by introducing an acidic composition at such a site can be avoided.

Certain related embodiments thus provide previously unforeseen advantages associated with pharmaceutical formulations containing both a first composition comprising at least one quinol compound having a first desired pharmacological activity, for instance, a catecholamine such as epinephrine, and a second active drug compound that comprises an amine group that is capable of being reversibly protonated at physiological pH, such a second active drug compound having a second desired pharmacological activity, which may be the same or different from the first desired pharmacological activity. Preferably the first and second desired pharmacological activities are beneficially co-administered. For example, the combination of a vasoconstrictor (such as the quinol compound epinephrine) with a local anesthetic that is capable of reversibly binding to a voltage-gated Na⁺ channel in a cell membrane (such as the amino amide anesthetic lidocaine) provides advantageously complementary pharmacological properties (which in the present example are believed according to non-limiting theory to include improved retention of the local anesthetic at or near the site of injection, by virtue of decreased dilution due to the vasoconstriction caused by epinephrine).

A surprising and heretofore unrecognized benefit afforded by certain invention embodiments disclosed herein is that stabilized quinol compositions and/or stable pharmaceutical formulations may be administered that have pH values closer to physiological pH values than previously contemplated, in a manner that both reduces the level of pain experienced by the recipient upon parenteral administration (e.g., hypodermic injection), and that facilitates transport across cell membranes of compound(s) having desired pharmacological activities, such as active drug ingredients having amine groups that can be reversibly protonated. According to non-limiting theory, such benefits derive from (a) reduced pain that results from neutral and not acidic pH of the administered solution, and (b) improved transmembrane transit of a pharmacologically active drug ingredient having an amine group that is capable of being reversibly protonated, which results from maintenance by neutral or near-neutral pH of amine groups in the unprotonated, and therefore uncharged, form at the point where the drug crosses the hydrophobic interior of the plasma membrane. Hence, and further according to theory, more rapid manifestation of a desired pharmacological effect is made possible by these embodiments.

Accordingly and as described in greater detail below, the invention thus derives from the discovery of heretofore unappreciated benefits to be obtained by exploiting a combination of interactions among established principles of chemical equilibrium, namely the interplay of (i) an effect of pH on the stability of quinol compounds to avoid oxidative damage, (ii) antioxidant protective effects for quinol compounds conferred by the presence of thiol agents, based on increased reducing potentials for thiol agents at pH levels that are closer to neutrality than the acidic pH levels employed by the prior art in efforts to stabilize quinol compounds, (iii) dramatic effects on thiol agents' reducing potential induced by modest increases in thiol agent concentrations, and (iv) selection of suitable pH buffers at suitable concentrations and having pKa values that are close to the pH value sought to be maintained.

The present invention is directed in pertinent part, according to certain herein disclosed embodiments, to a stabilized quinol composition comprising at least one isolated quinol compound, at least one thiol agent, and at least one pH buffer that maintains a substantially constant pH in the stabilized quinol composition. Quinol compounds include any of a large number of chemical compounds that comprise a quinol (dihydroxybenzene) moiety, and preferably such a quinol moiety is present as an orthoquinol or a paraquinol. Quinol compounds may undergo interconversion between quinol forms and oxidatively degraded quinone forms, and in preferred embodiments the quinol compound is maintained in the quinol form, which is the reduced form. Criteria for determining whether a quinol compound is present in reduced (quinol) or oxidized (quinone) form are known in the art, and may include, for example, spectrophotometric (e.g., colorimetric or ultraviolet absorbance) or spectroscopic (e.g., mass spectrometry or nuclear magnetic resonance spectroscopy) characterization of a sample, depending on the particular compound. As noted above, exemplary quinol compounds include the catecholamines (e.g., epinephrine, norepinephrine, levonordefrin; see, e.g., U.S. Pat. No. 5,002,973). In certain preferred embodiments the quinol compound has at least one desired pharmacological activity as provided herein. An exemplary quinol compound is epinephrine, and other examples of quinol compounds include 1,2-dihydroxybenzene (catechol, pyrocatechol), norepinephrine, dopamine, dobutamine, isoproterenol, racepinephrine, arbutamine, carbidopa, deoxyepinephrine, dioxethedrine, 3-(3,4-dihydroxyphenyl)-alanine (L-, D- or DL-DOPA), dopexamine, droxidopa, ethylnorepinephrine, hexoprenaline, isoetharine, methyldopa, N-methylepinephrine, nordefrin, rimiterol, epinephrine bitartrate, and L-epinephrine-D-hydrogentartate.

According to certain embodiments of the herein disclosed invention, a quinol compound comprises a compound of Formula (I):

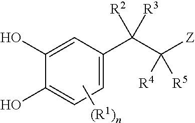

wherein:

n is 0, 1, 2 or 3

each R¹ is the same or different and independently hydrogen, alkyl, hydroxyl, alkoxide, —OC(O)alkyl, —OC(O)arylalkyl, arylalkyl, amino or halo;

R² and R³ are the same or different and independently hydrogen, hydroxyl, alkoxide, alkyl, oxo, —OC(O)alkyl, —OC(O)arylalkyl, amino, monoalkylamino, dialkylamino or halo;

R⁴ and R⁵ are the same or different and independently hydrogen, hydroxyl, alkoxide, —NR⁶₂, —NHNH₂ or lower alkyl,

Z is —NR⁶₂, —COOH or —CR⁷₃;

each R⁶ is the same or different and independently hydrogen, alkyl or arylalkyl; or

R⁵ and R⁶ together with the atoms to which they are attached form a heterocycle; and

each R⁷ is the same or different and independently hydrogen, alkyl, arylalkyl, —COOH, amino, —C(O)Oalkyl, —C(O)Oaryl, —C(O)Oarylalkyl, —NHNH₂, monoalkylamino or dialkylamino, as a single stereoisomer, a mixture of stereoisomers, or as a racemic mixture of stereoisomers, or as a pharmaceutically acceptable salt thereof.

“Alkyl” means an optionally substituted straight chain or branched, noncyclic or cyclic, unsaturated or saturated aliphatic hydrocarbon containing from 1 to 10 carbon atoms, while the term “lower alkyl” has the same meaning as alkyl but contains from 1 to 6 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like. Cyclic alkyls are also referred to herein as “homocycles” or “homocyclic rings.” Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Alkoxy” or “alkoxide” means an alkyl moiety attached through an oxygen bridge (i.e., —O-alkyl) such as methoxy, ethoxy, and the like.

“Alkylamino” and “dialkylamino” mean one or two alkyl moieties attached through a nitrogen bridge (i.e., —N-alkyl) such as methylamino, ethylamino, dimethylamino, diethylamino, and the like.

“Aryl” means an optionally substituted aromatic carbocyclic moiety such as phenyl or naphthyl.

“Arylalkyl” or “aralkyl” means an alkyl having at least one alkyl hydrogen atom replaced with an aryl moiety, such as benzyl, —(CH₂)₂ phenyl, —(CH₂)₃ phenyl, —CH(phenyl)₂, and the like.

“Halogen” or “halo” means fluoro, chloro, bromo and iodo.

“Haloalkyl” means an alkyl having at least one hydrogen atom replaced with halogen, such as trifluoromethyl and the like.

“Heterocycle” means a 5- to 7-membered monocyclic, or 7- to 10-membered bicyclic, heterocyclic ring which is either saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be optionally substituted with 1-5 substituents. The heterocycle may be attached to the rest of the molecule via any heteroatom or carbon atom. Examples of heterocycles include piperidinyl, 1,2,3,4-tetrahydroisoquinolinyl, 1,3-benzodioxolyl, morpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, imidazole, pyridine, indole, thiophene, benzopyranone, thiazole, furan, benzimidazole, quinoline, isoquinoline, quinoxaline, pyrimidine, pyrazine, tetrazole and pyrazole.

“Oxo” means a carbonyl (═O).

“Substituted” means any of the alkyl, aryl, arylalkyl or heterocycle wherein at least one hydrogen atom is replaced with a substituent. In the case of an oxo substituent (“═O”) two hydrogen atoms are replaced. Examples of suitable substituents include, but are not limited to, halogen, hydroxy, oxo, —COOH, alkyl, substituted alkyl, aryl, substituted aryl, heterocycle, substituted heterocycle, alkylamino and dialkylamino.

“Stable compound”, “stabilized formulation” and “stabilized composition” are meant to indicate a compound, formulation and/or composition that is sufficiently robust to survive isolation to a useful degree of purity from a reaction mixture, and formulation into an efficacious therapeutic agent. Preferably such a compound, formulation or composition can be stored at an ambient temperature for an extended time period, typically on the order of at least 4, 6, 8, 10, 12, 14, 16, 18, 20, 24, 26, 28, 30, 32, 34, 36, 38, 40, 42, 44, 46, 48, 50 or more months, while retaining most or substantially all of its pharmacological activity, typically at least 60%, 65%, 70%, 75%, 80%, 85%, 90%, 92%, 94%, 95%, 96%, 97%, 98%, 99% or more of at least one pharmacological activity as provided herein.

According to certain preferred embodiments, a stabilized quinol composition and/or a stable pharmaceutical formulation as provided herein may contain about 100% to about 105% of a desired pharmacological activity upon preparation according to the herein described methods, and may retain 90%, 92%, 94%, 95%, 96%, 97%, 98%, 99% or more of the desired pharmacological activity after storage at an ambient temperature for at least 4, 6, 8, 10, 12, 14, 16, 18, 20, 24, 26, 28, 30, 32, 34, 36, 38, 40, 42, 44, 46, 48, 50 or more months. These and related embodiments offer advantageously improved stability to afford increased precision in the delivery of a desired level of pharmacological activity, thereby providing enhanced safety and efficacy relative to other known preparations.

For instance, presently known epinephrine formulations may be initially prepared with, e.g., up to about 115% of a stated pharmacological activity in an effort to provide a specified amount of such activity even after degradation associated with storage over time, but instabilities such as oxidative and/or racemic degradation (as discussed above) may lead to loss of activity at a rate that is difficult to predict. Uncertainties may thus arise with respect to the actual amount of pharmacological activity residing in a preparation at the time of use, and such preparations must typically be labeled with expiration dates reflecting short shelf-lives. By contrast, the presently disclosed compositions and methods provide unprecedented improvements in the reliable delivery of desired pharmacological activity even following lengthy storage periods. Preferred quinol compositions and pharmaceutical formulations disclosed herein comprise an isolated quinol compound, a thiol agent and a pH buffer in quantities relative to one another so as to be suitable for pharmaceutical uses, including the pH buffer in an amount that provides buffering capacity capable of maintaining a substantially constant pH as described herein. Pharmacological activity may be determined by an appropriate activity assay as will be known to the skilled artisan depending on the particular composition for which maintenance of such a pharmacological activity is desired.

As used herein and in the appended claims, the singular forms “a,” “and,” and “the” include plural referents unless the context clearly dictates otherwise. Thus, for example, reference to “an agent” or to “a composition” includes a plurality of such agents or compositions, and reference to “the cell” includes reference to one or more cells and equivalents thereof known to those skilled in the art, and so forth. The term “comprising” (and related terms such as “comprise” or “comprises” or “having” or “including”) is not intended to exclude embodiments wherein, for example, any composition of matter, composition, method, or process, or the like, described herein may “consist of” or “consist essentially of” the described features.

According to certain embodiments, an isolated compound may be present, such as an “isolated” quinol compound, where the term “isolated” means that the material is removed from its original environment (e.g., the natural environment if it is naturally occurring). For example, a naturally occurring quinol compound that is present in an intact cell or tissue, or in a living plant or animal, is not isolated, but the same compound, separated from some or all of the co-existing materials in the natural system, is isolated. Such quinol compounds could be part of a crude extract or other man-made composition, and still be isolated in that such extract or composition is not part of its natural environment.

Additional examples of suitable quinol compounds for use in certain embodiments of the herein disclosed invention are presented in Table 1.

TABLE 1
Exemplary Quinol Compounds

| Compound | CAS# | Properties/Uses |
|---|---|---|
| Adrenalone | 99-45-6 | Hemostatic |
| Arbutamine | 128470-16-6 | Stress agent for coronary heart disease. |
| Benserazide | 322-35-0 | In combination with levodopa for antiparkinsonian |
| Carbidopa | 28860-95-9 | In combination with levodopa for antiparkinsonian |
| Deoxyepinephrine | 501-15-5 | hydrochloride as an adrenergic; vasoconstrictor |
| Dioxethdrine | 497-75-6 | Bronchodilator |
| Dobutamine | 34368-04-02 | Cardiotonic |
| Dopa | 63-84-3 | Antiparkinsonian |
| Dopamine | 51-61-6 | Cardiotonic; Antihypotensive |
| Dopexamine | 86197-47-9 | Cardiotonic |
| Droxidopa | 23651-95-8 | Antiparkinsonian |
| Epinephrine | 51-43-4 | Bronchodilator, cardiostimulant, mydriatic, antiglaucoma |
| Ethylnorepinephrine | 536-24-3 | Bronchodilator |
| Fluorodopa | 92812-82-3 | PET imaging |
| Hexoprenaline | 3215-70-1 | Bronchodilator, tocolytic |
| Isoetharine | 530-08-5 | Bronchodilator |
| Isoproterenol | 7683-59-2 | Bronchodilator, Sympathomimetic |
| Levodopa | 59-92-7 | Antiparkinsonian |
| Methyldopa | 555-30-6 | Antihypertensive |
| N-Methylepinephrine | 554-99-4 | Adrenergic |
| Nordefrin | 6539-57-7 | Vasoconstrictor |
| Norepinephrine | 51-41-2 | Adrenergic (vasopressor); antihypotensive. |
| | | Sympathomimetic; vasopressor in shock. |
| Protokylol | 136-70-9 | Bronchodilator |
| Rimiterol | 32953-89-2 | Bronchodilator |
| Tetrahydropapaveroline | 4747-99-3 | research tool in neurochemistry |

In certain preferred embodiments disclosed herein as may relate to a stabilized quinol composition and/or a stable pharmaceutical formulation, an isolated quinol compound may be present at from about at least 0.1% to about at least 2.0% weight-to-volume (w/v), i.e., by weight relative to the liquid volume of the composition or formulation (e.g., as an aqueous solution) such as about 0.2% (w/v), 0.4% (w/v), 0.6% (w/v), 0.8% (w/v), 0.9% (w/v), 1% (w/v), 1.2%(w/v), 1.4%(w/v), 1.5%(w/v), 1.6%(w/v), 1.7%(w/v), 1.8%(w/v) or 1.9%(w/v), for instance, at least from about 0.05 percent weight-to-volume to about 0.5 percent weight-to-volume, at least from about 0.01 percent weight-to-volume to about one percent weight-to-volume, at least from about 0.5 percent weight-to-volume to about two percent weight-to-volume, at least from about 0.75 percent weight-to-volume to about three percent weight-to-volume, at least from about one percent weight-to-volume to about four percent weight-to-volume, at least from about two percent weight-to-volume to about five percent weight-to-volume, or at least from about 2.5 percent weight-to-volume to about six percent weight-to-volume. For example, certain stabilized quinol compositions such as may be useful in ophthalmic applications (e.g., as eye drops) may comprise 2% (w/v) of an isolated quinol compound.

As described herein, certain embodiments relate to stabilized quinol compositions and/or stable pharmaceutical formulations that include one or more pharmacologically active compositions having at least one (i.e., one or more) pharmacological activity, which is preferably a desired pharmacological activity for purposes of beneficially administering such a composition to a human or animal. The pharmacological activity may be the result of a known mechanism of action of the composition or a constituent thereof (including, for example, a drug that is generated after administration of a prodrug) or may be the result of an unknown mechanism of action, where in either case pharmacological activity can be determined according to established criteria (e.g., demonstrable clinical benefit, altered biological or physiological properties in an art-accepted animal model etc.).

A pharmacological activity is understood to include an ability of an introduced composition of matter, such as a quinol compound having a first desired pharmacological activity and/or an amine-containing compound having a second desired pharmacological activity and at least one amine group that is capable of being reversibly protonated (e.g., a local anesthetic) to alter (i.e., increase or decrease in a statistically significant manner relative to an appropriate control) at least one biological or physiological function or structure in a host organism, preferably an animal such as a mammal, more preferably a primate and still more preferably a human.

For instance, the biological or physiological function or structure may include, but need not be limited to, any detectable parameter that directly relates to a condition, process, pathway, dynamic structure, state or other activity involving one or more cells, tissues, organs, systems (e.g., peripheral nervous system, central nervous system, musculoskeletal system, circulatory system, etc.) and/or interactions among such elements and for which altered (e.g., increased or decreased with statistical significance) function can be detected. The compositions and methods of certain embodiments of the present invention thus pertain in part to such correlation where the altered activity of a cell, tissue, organ and/or system may be, for example, an alteration in cell signal transduction including but not limited to nerve impulse or neuronal action potential conductance, and/or an alteration in cellular gene expression, and/or an alteration in cell membrane permeability, including selective permeability such as altered passive or active transport of physiologically relevant ions (e.g., Ca²⁺, Cl⁻, Na⁺, K⁺, Mg²⁺, etc.) metabolites, catabolites, precursors, cofactors, mediators and the like, and/or altered biosynthetic activity or degradation or shedding or release or uptake of one or more cellular or secreted components, or other criteria as provided herein and known to the relevant art.

A desired pharmacological activity includes a clinically beneficial or otherwise desirable effect and may have its origin in interactions of an administered composition with extracellular structures or events and/or with intramembranous or intracellular structures or events, in direct interactions with cellular genes and/or their gene products, or in structural or functional changes that occur as the result of interactions between intermediates that may be formed as the result of such interactions, including intermediates such as physiologically relevant ions (e.g., Ca²⁺, Cl⁻, Na⁺, K⁺, Mg²⁺, etc.), metabolites, catabolites, substrates, precursors, cofactors and the like. Additionally, pharmacological activity may include altered respiratory, metabolic or other biochemical or biophysical activity in some or all cells.

Accordingly, by way of non-limiting example, in certain herein disclosed embodiments a stable pharmaceutical formulation may comprise (i) a first composition that comprises at least one quinol compound that is epinephrine, having a first desired pharmacological activity that is vasoconstrictor activity, and (ii) a second composition that comprises at least one amine-containing compound having at least one amine group that is capable of being reversibly protonated that is lidocaine having a second desired pharmacological activity that is anesthetic activity as may result from reversible binding of lidocaine to a voltage-gated Na⁺ channel in a neuronal cell membrane, thereby altering Na⁺ movement through the voltage-gated channel. Persons familiar with the relevant art will appreciate that other quinol compounds and/or other reversibly protonated amine compounds (e.g., local anesthetics) may have other desired pharmacological activities, including such activities that may result when one or more quinol compounds are combined with one or more reversibly protonated amine compounds, such as in certain herein described stable pharmaceutical formulations.

Certain embodiments may thus relate to first and second pharmacological activities of, respectively, first and second compositions that are present within a stable pharmaceutical formulation. Such first and second pharmacological activities may be the same in certain embodiments, and in certain other embodiments the first and second pharmacological activities may be different from one another.

The presently disclosed stabilized quinol compositions and stable pharmaceutical formulations include at least one thiol agent, which refers to a compound possessing a functional group comprised of a sulfur atom and a hydrogen atom, which functional group may also be known as a sulfhydryl group, and which may also be present as a thiolate anion, depending on, or as a function of, the ambient pH. Thiol agents, which have long been known to have activity as reducing agents (e.g., Cleland, 1964 Biochem. 3:480), may also be known as mercaptans. In certain embodiments provided herein, at least one thiol agent may be present, for example, a thiol agent that is selected from cysteine, N-acetylcysteine, glutathione, monothioglycerol, cysteine ethyl ester, homocysteine, Coenzyme A, dithiothreitol, 2-mercaptoethanol, 2,3-dimercapto-1-propanol, 2,3-butanedithiol, 2-mercaptoethylamine, ethanedithiol, propanedithiol, 3-mercapto-2-butanol, dimercapto-propane-1-sulfonic acid, dimercaptosuccinic acid, trithiocyanuric acid, 2,5-dimercapto-1,3,4-thiadiazole, 3,4-dimercaptotoluene, 1,4-dimercapto-2,3-butanediol, 1,3-propanedithiol, 1,4-butanedithiol, and any thiol agent shown in Table 2.

TABLE 2
Additional Thiol Agents

| Name | CAS Registry Number |
|---|---|
| N-acetylpenicillamine | 15537-71-0 |
| ACV | 32467-88-2 |
| N-Amyl Mercaptan | 110-66-7 |
| Bucillamine | 65002-17-7 |
| N-Butyl Mercaptan | 109-79-5 |
| Sec-Butyl Mercaptan | 513-53-1 |
| Tert-Butyl Mercaptan | 75-66-1 |
| Captopril | 62571-86-2 |
| Cysteamine | 60-23-1 |
| DBHBT | 63147-28-4 |
| 2,3-Dimercapto-1-propanesulfonic acid | 74-61-3 |
| Dimercaprol | 59-52-9 |
| Dithiosalicylic Acid | 527-89-9 |
| 1,2-Ethanedithiol | 540-63-6 |
| Ethanethiol | 75-08-1 |
| Isobutyl Mercaptan | 513-44-0 |
| Mecysteine | 18598-63-5 |
| 2-Mercaptoethanol | 60-24-2 |
| MESNA | 19767-45-4 |
| Methanethiol | 74-93-1 |
| Pantetheine | 496-65-1 |
| Penicillamine | 52-67-5 |
| 1,3-propanedithiol | 109-80-8 |
| Succimer | 304-55-2 |
| Thioacetic Acid | 507-09-5 |
| Thiobenzyl Alcohol | 100-53-8 |
| Thiocyanic Acid | 463-56-9 |
| Thioglycerol | 96-27-5 |
| Thioglycolic Acid | 68-11-1 |
| Thiolactic Acid | 79-42-5 |
| Thiomalic Acid | 70-49-5 |
| Thionalide | 93-42-5 |
| 1-Thiosorbitol | 24531-57-5 |
| Tiopronin | 1953-02-2 |
| Tixocortol | 61951-99-3 |
| Trithiocarbonic Acid | 594-08-1 |

In certain preferred embodiments the thiol agent may be at least one of cysteine, acetylcysteine, glutathione, cysteine ethyl ester and monothioglycerol. Preferably, the thiol agent is present at a concentration that is in molar excess relative to the concentration of the quinol compound. One or more thiol agents are thus expected to be present at a concentration that is capable of maintaining a suitable reducing potential, where the degree of molar excess and the reducing potential may be determined by a person skilled in the art and based on the disclosure herein, as a function of the quinol compound that is used, of the molecular weight of the thiol agent that is selected, and of the pH. Typically these or other thiol agents as provided herein may be used at working concentrations of at least 0.1 to 10 mg/ml or at higher concentrations such as at least 15, 20, 25, 30, 35, 40, 45 or 50 mg/ml, with the thiol agent being present at a concentration of at least 1-10 mg/ml in certain preferred embodiments and the thiol agent being present at a concentration of at least 1-5 mg/ml in certain other preferred embodiments

According to non-limiting theory, the present invention exploits heretofore unappreciated interactions between (i) the quantitative relationship among pH, the pKa of a buffer (i.e., an acid) and the concentrations of the buffer's proton-donor and proton-acceptor species (e.g., the relationship described by the Henderson-Hasselbalch equation) and (ii) the quantitative relationship among the redox potential of a redox couple (i.e., a thiol agent) and the concentrations of a thiol agent's electron-donor (i.e., sulfhydryl) and electron-acceptor (i.e., disulfide) species (e.g., the relationship described by the Nernst equation), in the context of stabilization of a quinol compound.

Further, and without wishing to be bound by theory, thiol agents are capable of reversibly reacting to form disulfide linkages with the accompanying generation of two hydrogen ions per disulfide formed, i.e., a decrease in pH. Hence, when pH increases, as is the case when hydrogen ion concentration decreases as a consequence of the combined effects of hydrogen ion neutralization by the pH buffer and disulfide reduction to free sulfhydryls, an increase in the available reducing potential provided by the thiol agent results.

Accordingly, in a stabilized quinol composition comprising a quinol compound and a pH buffer, inclusion of at least one thiol agent in molar excess relative to the quinol compound is thus believed to contribute to the stability of the herein described quinol compositions and pharmaceutical formulations, as a result of the relationship between the solution pH and its effect (i.e., the effect of hydrogen ion concentration) on the reduction potential of thiols. Briefly, because of their antioxidant properties, inclusion of a molar excess of thiol groups in a solution containing a quinol compound is believed to create a reducing environment that prevents oxidation of the quinol to a quinone. Contrary to prior art teachings that low (acidic, e.g., pH<4) pH values are important to stabilization of quinol compounds (e.g., U.S. Pat. No. 6,008,256), it is herein disclosed for the first time that at a relatively elevated pH (e.g., pH 5.5-9) such as a substantially constant pH that is maintained by a pH buffer, the presence of one or more thiol agents may protect quinols from oxidation that would otherwise proceed readily in the absence of thiols. Certain herein disclosed embodiments expressly contemplate formulations that do not contain any sulfite and/or to which no sulfite is added.

According to certain embodiments the herein described stabilized quinol compositions and stable pharmaceutical formulations comprise a pH buffer which is present under conditions and in sufficient quantity to maintain a pH. A suitable pH buffer may be selected for desired properties according to criteria disclosed herein and known to those skilled in the art, depending on the particular intended use in a stabilized quinol composition and/or a stable pharmaceutical formulation. These criteria include the pKa of the buffer, the pH that will be desirably maintained, the capacity and pH range of the buffer, the temperature at which the buffer will be used, chemical compatibility of the buffer with one or more of the quinol compound (e.g., a catecholamine), the thiol agent, the pharmacologically active amine-containing compound (e.g., a local anesthetic) and/or other components of the stabilized quinol composition or stable pharmaceutical formulations, the safety and toxicity profiles of the buffer, and other criteria.

In certain related embodiments the pH buffer is present under conditions and in sufficient quantity to maintain a pH that is “about” a recited pH value. “About” such a pH refers to the functional presence of that buffer, which, as is known in the art, may be a consequence of a variety of factors including pKa value(s) of the buffer, buffer concentration, working temperature, effects of other components of the composition on pKa (i.e., the pH at which the buffer is at equilibrium between protonated and deprotonated forms, typically the center of the effective buffering range of pH values), and other factors.

Hence, “about” in the context of pH may be understood to represent a quantitative variation in pH that may be more or less than the recited value by no more than 0.5 pH units, more preferably no more than 0.4 pH units, more preferably no more than 0.3 pH units, still more preferably no more than 0.2 pH units, and most preferably no more than 0.1-0.15 pH units. As also noted above, in certain embodiments a substantially constant pH (e.g., a pH that is maintained within the recited range for an extended time period) may be from about pH 5.5 to about pH 9, from about pH 5.5 to about pH 8.5, from about pH 5.5 to about pH 8.25, from about pH 5.5 to about pH 8.0, from about pH 5.75 to about pH 7.75, or from about pH 6.0 to about pH 7.5, or any other pH or pH range as described herein. As also noted above, maintenance of a substantially constant pH preferably includes an ability to regulate the pH of the composition or formulation so that it remains at “about” a recited pH for a lengthy period of time, typically on the order of at least 4, 6, 8, 10, 12, 14, 16, 18, 20, 24 or more months. Similarly, in the context of other quantitative parameters “about” may be understood to reflect a quantitative variation that may be more or less than the recited value by 0.5 logarithmic units (e.g., “logs” or orders of magnitude), more preferably no more than 0.4 log units, more preferably no more than 0.3 log units, still more preferably no more than 0.2 log units, and most preferably no more than 0.1-0.15 log units.

Therefore the pH buffer typically may comprise a composition that, when present under appropriate conditions and in sufficient quantity, is capable of maintaining a desired pH level as may be selected by those familiar with the art, for example, buffers comprising phosphate (e.g., monobasic and/or dibasic sodium phosphate or potassium phosphate), Tris, Tricine, citrate, acetate, other phosphate salts, borate, carbonate and/or bicarbonate, HEPES, HEPPS, MES, MOPS, ACES, imidazole, diethylmalonic acid, PIPES, TES, or other buffers such as those in Table 2 or known to the art (see, e.g., Calbiochem® Biochemicals & Immunochemicals Catalog 2004/2005, pp. 68-69 and catalog pages cited therein, EMD Biosciences, La Jolla, Calif.) and suitable solutes such as salts (e.g., KCl, NaCl, CaCl₂, MgCl₂, etc.) for maintaining, preserving, enhancing, protecting or otherwise promoting desired biological or pharmacological activity of a quinol compound and/or of an amine-containing compound such as an active drug ingredient that may be present in the herein described stable quinol composition and/or in the presently disclosed stable pharmaceutical formulation or other components, for instance, phosphate, acetate, sodium chloride/sodium citrate buffer (SSC), MOPS/sodium acetate/EDTA buffer (MOPS), ethylenediamine tetraacetic acid (EDTA), sodium acetate buffer at physiological pH, and the like. See, e.g., Remington's Pharmaceutical Sciences, Mack Publishing Co. (A.R. Gennaro edit. 1985). Certain herein disclosed embodiments expressly contemplate formulations that do not contain any borate, citrate, lactate and/or cysteine, and/or to which no borate, citrate, lactate and/or cysteine is added.

TABLE 3
Exemplary Buffers

| Buffer | pKa |
|---|---|
| Citric acid (pKa2) | 4.76 |
| Malate (pKa2) | 5.13 |
| Pyridine | 5.23 |
| Piperazine (pKa1) | 5.33 |
| Succinate (pKa2) | 5.64 |
| Histidine | 6.04 |
| Maleate (pKa2) | 6.24 |
| Citric acid (pKₐ₃) | 6.40 |
| Bis-Tris | 6.46 |
| Pyrophosphate (pKa3) | 6.70 |
| PIPES | 6.76 |
| ACES | 6.78 |
| Histidine | 6.80 |
| MOPSO | 6.87 |
| Imidazole | 6.95 |
| BES | 7.09 |
| MOPS | 7.14 |
| HEPES | 7.48 |
| DIPSO | 7.52 |
| MOBS | 7.60 |
| TAPSO | 7.61 |
| Triethanolamine | 7.76 |
| POPSO | 7.78 |
| Tricine | 8.05 |
| MES | 6.15 |
| Cacodylic acid | 6.27 |
| H₂CO₃/NaHCO₃ (pKₐ₁) | 6.37 |
| ADA | 6.60 |
| Bis-Tris Propane (pKₐ₁) | 6.80 |
| NaH₂PO₄/Na₂HPO₄ (pKₐ₂) | 7.21 |
| TES | 7.50 |
| HEPPSO | 7.85 |

Pharmaceutical Actives Including Local Anesthetics

Certain preferred embodiments of the invention disclosed herein relate to a stable pharmaceutical formulation comprising a first composition that comprises at least one quinol compound having a first desired pharmacological activity; a second composition that comprises at least one amine-containing compound having (i) at least one amine group that is capable of being reversibly protonated, and (ii) a second desired pharmacological activity; at least one thiol agent; and at least one pH buffer that maintains a substantially constant pH in the pharmaceutical formulation. The second composition comprising a pharmacologically active compound and having at least one amine group that is capable of being reversibly protonated may in certain embodiments comprise an amine group having a pKa of from about pH 7.5 to about pH 9.3, from about pH 7.6 to about pH 9.2, from about pH 7.7 to about pH 9.1, from about pH 7.8 to about pH 9.0, from about pH 7.9 to about pH 8.9, from about pH 8.0 to about pH 8.8, from about pH 8.1 to about pH 8.7, from about pH 8.2 to about pH 8.6, or from about pH 8.3 to about pH 8.5, and in other embodiments the amine group may have a distinct pKa value.

Certain preferred embodiments contemplate a pharmacologically active, reversibly protonated amine-containing compound that is a local anesthetic, such as an amino ester local anesthetic (e.g., procaine) or an amino amide local anesthetics (e.g., lidocaine). See, e.g., Rang, Dale, Ritter and Moore, Pharmacology (Fifth Edition), Churchill Livingstone/Elsevier Ltd., London, 2003, Ch. 43, pages 612-618. According to these and related embodiments, the local anesthetic compound is capable of reversibly binding to a voltage-gated Na⁺ channel in a cell membrane, and in many such embodiments a local anesthetic compound may comprise at least one amine group that is capable of being reversibly protonated. (e.g., Hille, 1992 Ionic Channels of Excitable Membranes, Sinauer, Sunderland, Mass.; Strichartz et al. 1987 In Local Anesthetics—Handbook of Experimental Pharmacology (G. R. Strichartz, Ed.), 81:21-52. Such binding may alter (e.g., increase or decrease in a statistically significant manner) Na⁺ movement through the voltage-gated Na⁺ channel.

Preferred embodiments contemplate a local anesthetic that reversibly but substantially blocks (e.g., decreases in a statistically significant manner by at least 25%, 40%, 50%, 60%, 70%, 80%, 90%, 95%, 96%, 97%, 98% or 99% relative to when the local anesthetic is not present) the movement of Na⁺ ions through the voltage-gated Na⁺ channel. According to certain other embodiments a stable pharmaceutical formulation may comprise a first composition that comprises at least one quinol compound having a first desired pharmacological activity; a second composition that comprises lidocaine; at least one thiol agent; and at least one pH buffer that maintains a substantially constant pH in the pharmaceutical formulation.

Non-limiting examples of these and other local anesthetics that may be usefully employed as the second composition in the above described stable pharmaceutical formulation are presented in Table 4 and in Rang et al., (2003, supra), Hille (1992, supra), Strichartz (1987, supra) and Ragsdale et al., 1994 Science 265:1724. As also noted above, these and related invention embodiments provide unexpected advantages associated with obtaining the benefits of co-administering a quinol compound and a local anesthetic, such as a vasoconstrictor (e.g., epinephrine) and lidocaine, at a pH that, by being closer to physiological pH than the acidic pH formulations previously used for catecholamine-containing formulations, facilitates cellular uptake of the anesthetic and reduces the level of pain associated with local injection.

Accordingly, and in related embodiments, a local anesthetic may comprise an amino amide linkage (e.g., —(C═O)—O—, as found in procaine, cocaine, tetracaine, dibucaine) or an amino ester linkage (e.g., —NH—(C═O)—, as found in lidocaine, prilocalne, bupivacaine) that is situated between (i) the amine group that is capable of being reversibly protonated and (ii) a lipophilic moiety, where “lipophilic moiety” is intended to refer to moieties which are hydrophobic and/or lipophilic in nature. Advantageously, such moieties contribute to the ability of the local anesthetic to be non-selectively permeable in the lipid bilayer of a cell membrane such that the local anesthetic can access intramembranous sites of its target molecule(s), such as a transmembrane domain of a voltage-gated Na⁺ channel.

Examples of hydrophobic/lipophilic moieties include aryl, aralkyl, aryl heterocycles, polycycyls, and heterocyclyls, such as benzene, pyrrole, furan, thiophene, imidazole, oxazole, thiazole, triazole, pyrazole, pyridine, pyrazine, pyridazine and pyrimidine, naphthyl, quinolyl, indolyl, tetralin, pyrrolidine, oxolane, thiolane, oxazole, piperidine, piperazine, morpholine, lactones, lactams, pyrrolidinones, lactones, sultams, and sultones. A preferred lipophilic moiety is benzyl or a benzyl derivative that provides an aromatic ring system, such as those found in lidocaine, Xylocalne, mepivacaine, Carbocaine, Isocaine, bupivacaine, Marcaine, etidocaine, prilocalne, cocaine, procaine, Novocain, tetracaine, Pontocaine and benzocaine.

Preferred local anesthetic compounds are capable of reversibly binding to a voltage-gated Na⁺ channel in a cell membrane, such as a plasma membrane, for a time and in a manner sufficient to block Na⁺ movement through the channel. For example, when the cell is a neuron, such Na⁺ blockade blocks action potential along the neuron and blocks neural conduction, e.g., of neuronal signals. Local anesthetics may additionally or alternatively bind to a voltage-gated Na⁺ channel in a cell membrane in a striated muscle cell or in a cardiac myocyte. A voltage-gated Na⁺ channel may be present in a plasma membrane or in another cell membrane, such as an endoplasmic reticulum membrane, a mitochondrial membrane (e.g., inner mitochondrial membrane), a lysozomal membrane, an exocytic vacuolar membrane, an endocytic vacuolar membrane, or the like, or in a manipulated or non-naturally occurring membrane such as a liposome, a microsome, a membrane vesicle, or the like.

It will be important to note that these and related embodiments, in which the second composition comprises at least one local anesthetic compound that is capable of reversibly binding to a voltage-gated Na⁺ channel in a cell membrane to thereby alter Na⁺ movement through the voltage-gated Na⁺ channel, expressly exclude combinations in which a noradrenergic neuron-blocking drug that directly blocks noradrenaline release from neurons, such as guanethidine, betanidine, bretylium or the like (see, e.g., Rang et al., Pharmacology (Fifth Edition), Churchill Livingstone/Elsevier Ltd., London, 2003, chapter 11, page 177; Clough et al., U.S. Pat. No. 4,164,570) is used in place of the second composition that is capable of binding to a voltage-gated Na⁺ channel in a cell, insofar as such noradrenergic neuron-blocking drugs are not capable of binding to a voltage-gated Na⁺ channel in a cell. In certain preferred embodiments, therefore, local anesthetics as described herein do not directly displace norepinephrine in a peripheral neuron, in contrast to noradrenergic neuron-blocking drugs that directly block noradrenaline release from neurons by displacing the natural intracellular catecholamine pools such as are found at nerve endings.

TABLE 4
Exemplary Local Anesthetics

| | pKa | % RNH(+) | % RN | % RNH(+) | % RN | % RNH(+) | % RN |
|---|---|---|---|---|---|---|---|
| Anesthetic | (amine) | at pH 7.4 | at pH 7.4 | at pH 6.5 | at pH 6.5 | at pH 5.0 | at pH 5.0 |
| Benzocaine | 3 | 0.00 | 100.00 | 0.03 | 99.97 | 0.99 | 99.01 |
| Mepivacaine | 7.6 | 61.31 | 38.69 | 92.64 | 7.36 | 99.75 | 0.25 |
| Etidocaine | 7.7 | 66.61 | 33.39 | 94.06 | 5.94 | 99.80 | 0.20 |
| Articaine | 7.8 | 71.53 | 28.47 | 95.23 | 4.77 | 99.84 | 0.16 |
| Lidocaine | 7.9 | 75.97 | 24.03 | 96.17 | 3.83 | 99.87 | 0.13 |
| Prilocaine | 7.9 | 75.97 | 24.03 | 96.17 | 3.83 | 99.87 | 0.13 |
| Bupivacaine | 8.1 | 83.37 | 16.63 | 97.55 | 2.45 | 99.92 | 0.08 |
| Ropivacaine | 8.1 | 83.37 | 16.63 | 97.55 | 2.45 | 99.92 | 0.08 |
| Tetracaine | 8.2 | 86.32 | 13.68 | 98.04 | 1.96 | 99.94 | 0.06 |
| Cocaine | 8.6 | 94.06 | 5.94 | 99.21 | 0.79 | 99.97 | 0.03 |
| Chloroprocaine | 8.7 | 95.23 | 4.77 | 99.37 | 0.63 | 99.98 | 0.02 |
| Dibucaine | 8.8 | 96.17 | 3.83 | 99.50 | 0.50 | 99.98 | 0.02 |
| Procaine | 8.9 | 96.93 | 3.07 | 99.60 | 0.40 | 99.99 | 0.01 |
| Hexylcaine | | | | | | | |
| Piperocaine | | | | | | | |
| Propoxycaine | | | | | | | |

According to certain related embodiments, the first composition (i.e., the quinol compound) may be present in the stable pharmaceutical formulation at a mass-to-volume ratio in the formulation that is between about 1:500,000 and about 1:10,000, such as 1:2.5×10⁵, 1:2×10⁵, 1:1.5×10⁵, 1:1.25×10⁵, 1:1×10⁵, 1:5×10⁴, 1:2.5×10⁴, 1:1×10⁴, or another ratio within this range.

Also contemplated within certain embodiments of the present invention are methods of stabilizing a quinol compound as provided herein, and methods of stabilizing a pharmaceutical formulation as provided herein, the methods comprising contacting the recited components according to procedures and practices that will be apparent to those having familiarity with the relevant art (e.g., good manufacturing practices, “GMP”). Thus, a method of stabilizing a quinol compound is provided, comprising contacting at least one isolated quinol compound, at least one thiol agent, and a pH buffer that maintains a substantially constant pH, under conditions and for a time sufficient to stabilize the compound. Similarly, there is provided a method of stabilizing a pharmaceutical formulation, comprising contacting a pharmaceutical formulation, at least one thiol agent, and a pH buffer that maintains a substantially constant pH, under conditions and for a time sufficient to stabilize the pharmaceutical formulation, which comprises (i) a first composition that comprises at least one quinol compound having a desired pharmacological activity, and (ii) a second composition that comprises at least one amine-containing compound having at least one amine group that is capable of being reversibly protonated, such as a local anesthetic that is capable of reversibly binding to a voltage-gated Na⁺ channel in a cell membrane. Preferably the thiol agent is present in a molar excess relative to the quinol compound, and the pH buffer is provided under conditions and in sufficient quantity (e.g., at a sufficient concentration as determined, for example, using the Henderson-Hasselbalch equation) to maintain a desired pH that is substantially constant.

These and related methods provided herein for the first time offer advantages in the production, handling and use of quinol compositions, as may be associated with increased convenience and reduced costs for shipping, maintaining inventory, and delivering such products to remote or undeveloped areas such as those lacking refrigeration or facilities for sterile reconstitution, and for making such products available for travel, outdoor recreation, emergency and military uses.

As noted above, according to certain preferred embodiments the herein described stabilized quinol compositions and/or pharmaceutical formulations may be prepared in solid form without added excipients, while according to certain other preferred embodiments the stabilized quinol compositions and/or pharmaceutical formulations may also comprise an acceptable pharmaceutical carrier. Such carriers will be nontoxic to recipients at the dosages and concentrations employed. For therapeutic agents, about 0.005 μg/kg to about 100 mg/kg body weight will be administered, typically by the intradermal, subcutaneous, intramuscular or intravenous route, or by other routes. A preferred dosage is about 0.1 μg/kg to about 1 mg/kg, with about 2.5 μg/kg to about 200 μg/kg particularly preferred. Preferred embodiments contemplate parenteral administration to a subject of the herein described stabilized quinol compositions and/or stable pharmaceutical formulations. The subject may be a human subject such as a patient in need of treatment, or a non-human animal, preferably a mammal, for whom administration of a quinol composition and/or of a pharmaceutical formulation may be indicated according to clinical or other suitable criteria known in the relevant art. For example, in the case of a stable pharmaceutical formulation comprising a quinol compound and a local anesthetic, selection of particular quinol compounds and of particular local anesthetics may vary as a function of anatomical site identified for administration, nature of the medical procedure, duration of desired pharmacological activity (e.g., drug effect), age and weight of the subject, and other factors.

Suitable dosages of first and second compositions within a pharmaceutical formulation as provided herein may be independent of one another in some embodiments, while in other embodiments the dosage of a first composition that comprises at least one quinol compound having a desired pharmacological activity may be lower in such a formulation (which also comprises a second composition that comprises at least one amine containing compound having at least one amine group that is capable of being reversibly protonated) than is the case in a stabilized quinol composition that lacks such a second composition. It will be evident to those skilled in the art that the number and frequency of administration will be dependent upon the response of the host.

“Pharmaceutically acceptable carriers” for therapeutic use are well known in the pharmaceutical art, and are described, for example, in Remington's Pharmaceutical Sciences, Mack Publishing Co. (A.R. Gennaro edit. 1985). For example, sterile saline and phosphate-buffered saline at physiological pH may be used, as may be other buffering systems described herein. Preservatives, stabilizers, dyes, antimicrobial agents and even flavoring agents may be provided in the pharmaceutical composition. For example, sodium benzoate, sorbic acid (2,4-hexadienoic acid) and esters of p-hydroxybenzoic acid may be added as preservatives. Id. at 1449. In addition, antioxidants and suspending agents may be used. Id. “Pharmaceutically acceptable salt” refers to salts of the compounds of the present invention derived from the combination of such compounds and an organic or inorganic acid (acid addition salts) or an organic or inorganic base (base addition salts). The compounds of the present invention may be used in either the free base or salt forms, with both forms being considered as being within the scope of the present invention. The pharmaceutical compositions that contain one or more quinol compounds and/or amine-containing compounds may be in any form that allows for the composition to be administered to a patient. For example, the composition may be in the form of a solid, liquid or gas (aerosol). Typical routes of administration include, without limitation, oral, topical (including ophthalmic), parenteral (e.g., sublingually or buccally), sublingual, rectal, vaginal, and intranasal. The term parenteral as used herein includes subcutaneous injections, intravenous, intramuscular, intrasternal, intracavernous, intrathecal, intrameatal, intraurethral injection or infusion techniques. The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of the invention in aerosol form may hold a plurality of dosage units.

For oral administration, an excipient and/or binder may be present. Examples are sucrose, kaolin, glycerin, starch dextrins, sodium alginate, carboxymethylcellulose and ethyl cellulose. Coloring and/or flavoring agents may be present. A coating shell may be employed. The composition may be in the form of a liquid, e.g., an elixir, syrup, solution, emulsion or suspension. The liquid may be for oral administration or for delivery by injection, as two examples. When intended for oral administration, preferred compositions contain, in addition to one or more quinol compounds and/or amine-containing compounds, one or more of a sweetening agent, preservatives, dye/colorant and flavor enhancer. In a composition intended to be administered by injection, one or more of a surfactant, preservative, antimicrobial agent, wetting agent, dispersing agent, suspending agent, buffer, stabilizer and isotonic agent may be included.

A liquid pharmaceutical composition as used herein, whether in the form of a solution, suspension or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile. It may also be desirable to include other components in the preparation, such as delivery vehicles including but not limited to aluminum salts, water-in-oil emulsions, biodegradable oil vehicles, oil-in-water emulsions, biodegradable microcapsules, and liposomes.

While any suitable carrier known to those of ordinary skill in the art may be employed in the pharmaceutical compositions of this invention, the type of carrier will vary depending on the mode of administration and whether a sustained release is desired. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109. In this regard, it is preferable that the microsphere be larger than approximately 25 μm.

Pharmaceutical compositions may also contain diluents such as buffers, antioxidants such as ascorbic acid, glutathione, low molecular weight (less than about 10 residues) polypeptides, proteins, amino acids, carbohydrates including glucose, sucrose or dextrins, chelating agents such as EDTA, EGTA, citric acid, and other stabilizers and excipients. Neutral buffered saline or saline mixed with nonspecific serum albumin are exemplary appropriate diluents. Preferably, product is formulated as a lyophilizate, without added excipients according to certain embodiments, and according to certain other embodiments, using appropriate excipient solutions (e.g., saline, sucrose) as diluents.

Preferred compositions and preparations are prepared so that a parenteral dosage unit contains between about 0.01% to about 1% by weight, about 0.1% to about 2%, or about 0.5 to about 5% of active compound.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, beeswax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Exemplary topical formulations may contain a concentration of the quinol compound(s) and/or amine-containing compound(s) of from about 0.1 to about 10% w/v (weight per unit volume). The composition may be intended for rectal administration, in the form, e.g., of a suppository which will melt in the rectum and release the drug. The composition for rectal administration may contain an oleaginous base as a suitable nonirritating excipient. Such bases include, without limitation, lanolin, cocoa butter and polyethylene glycol. In the methods of the invention, the pharmaceutical compositions comprising stabilized quinol compositions and/or stable pharmaceutical formulations of the present invention, and/or comprising one or more quinol compounds, thiol agents, pH buffers and/or amine-containing compounds as described herein, may be administered through use of insert(s), bead(s), timed-release formulation(s), patch(es) or fast-release formulation(s).

The following Examples are offered by way of illustration and not limitation.

### Example 1

### Stabilization of Epinephrine

This example describes stabilization of epinephrine, a quinol compound, in the presence of a thiol agent (cysteine) and a pH buffer that maintains a substantially constant pH. All reagents are available from Sigma-Aldrich (St. Louis, Mo.).

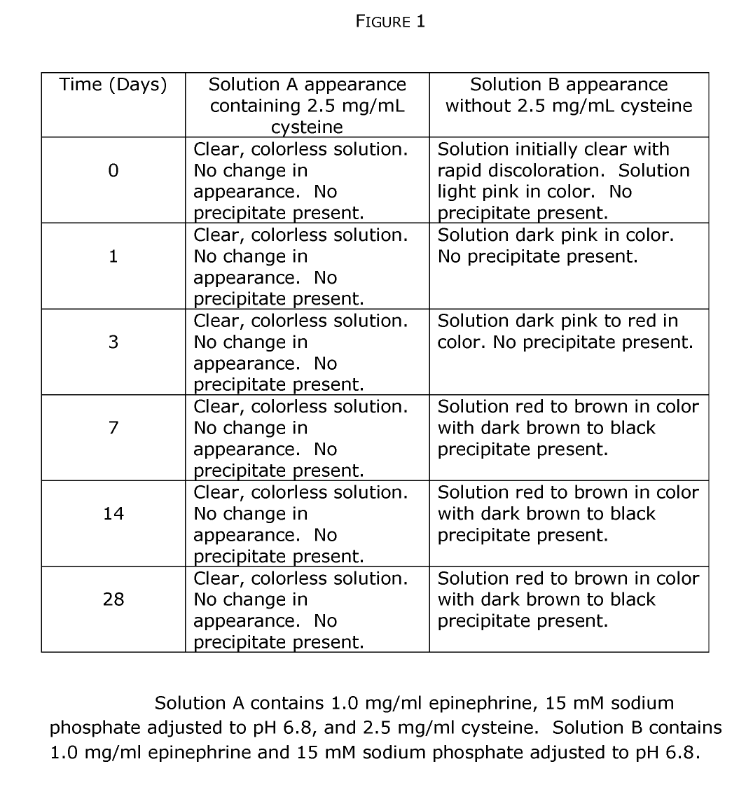

Under aseptic conditions a sterile aqueous solution of 15 mM sodium phosphate-pH 6.8 is prepared by admixing appropriate quantities of 15 mM NaH₂PO₄ and Na₂HPO₄. Into the sodium phosphate solution solid epinephrine bitartrate powder is dissolved to achieve a final epinephrine concentration of 1.0 mg/mL. The solution is divided into two equal portions and to one portion is added crystalline cysteine to a final concentration of 2.5 mg/mL. An aliquot of each solution is taken for immediate analysis of epinephrine degradation and the two solutions are immediately stored in the dark at room temperature in containers having airtight seals; the time is noted as the zero time point (t₀). At time points of 0, 1, 3, 7, 14, and 28 days aliquots are withdrawn for analysis (i) by visual inspection for color change, and/or (ii) by chromatographic and spectrometric determination of epinephrine degradation according to methodologies described in Robinson et al. (2000 Anesthesia 55:853), Newton et al. (1981 Am. J. Hosp. Pharm. 38:1314), Kalyanaraman et al. (1984 J. Biol. Chem. 259:354), Kirchhoefer et al. (1986 Am J. Hosp. Pharm. 43:1736; 1986 Am J. Hosp. Pharm. 43:1741), and/or Stepensky et al. (2004 J. Pharm Sci. 93:969), including references cited therein. Such an analysis is depicted, for example, in FIG. 1, for Solution A containing 2.5 mg/ml cysteine and Solution B without cysteine.

### Example 2

### Stabilized Epinephrine for Pharmaceutical Injection

A stabilized epinephrine formulation is compounded using the following methodology. Epinephrine bitartrate is added to a volumetric container to give a final concentration of 1.0 mg/ml of epinephrine base. Next, a sufficient quantity of cysteine is added to give a final concentration of 2.5 mg/ml. Next, sodium phosphate monobasic (NaH₂PO₄) and sodium phosphate dibasic (Na₂HPO₄) are added to the vessel in an equal molar ratio to give a final concentration of 15 mM. Next, a sufficient quantity of chlorobutanol (as an antimicrobial agent) is added to give a final concentration of 5.0 mg/ml. Next, Ethylenediaminetetraacetic Acid (EDTA) disodium is added to give a final concentration of 0.1 mM. The vessel is well mixed and water for injection (WFI) that has been previously sparged with an inert gas such as helium, argon, or nitrogen is added to the vessel in sufficient quantity to fill it to seventy-five percent of its final volume. The solution is then vigorously stirred for a sufficient time to solubilize and evenly distribute all ingredients. During this time a sufficient overlay of inert gas (e.g., N₂) is maintained to substantially prevent introduction of molecular oxygen into the system. Preferably the system is kept protected from sunlight, such as in an ambered vessel, and from other lights emitting ultraviolet light to inhibit photooxidation. The vessel also preferably excludes any metallic materials to minimize contamination with trace metals. The formulation is compounded aseptically under sterile conditions using ingredients suitable for their intended use. Next the solution is taken to ninety percent of its final volume and carefully adjusted to pH 6.8 with sterile 1.0 M HCl and/or 1.0 M NaOH solutions. The solution is then brought to ninety-five percent of its final volume and the isotonicity is adjusted to 290±20 mOsmolar using a sterile saturated sodium chloride solution or neat sodium chloride. The solution is then taken to its final volume with WFI and mixed thoroughly.

### Example 3

### Stabilized Epinephrine and Local Anesthetic Compound for Pharmaceutical Injection

A stabilized epinephrine formulation containing a local anesthetic comprising of at least one amine group that is capable of being reversibly protonated is compounded using the following methodology. Lidocaine hydrochloride is added to a volumetric container to give a final concentration of 10 mg/ml. Next, epinephrine bitartrate is added to the volumetric container to give a final concentration of 0.010 mg/ml of epinephrine base. Next, a sufficient quantity of cysteine is added to give a final concentration of 2.5 mg/ml. Next, sodium phosphate monobasic (NaH₂PO₄) and sodium phosphate dibasic (Na₂HPO₄) are added to the vessel in an equal molar ratio to give a final concentration of 15 mM. Next, a sufficient quantity of chlorobutanol (as an antimicrobial agent) is added to give a final concentration of 5.0 mg/ml. Next, Ethylenediaminetetraacetic Acid (EDTA) disodium is added to give a final concentration of 0.1 mM. The vessel is well mixed and water for injection (WFI) that has been previously sparged with an inert gas such as helium, argon, or nitrogen is added to the vessel in sufficient quantity to fill it to seventy-five percent of its final volume. The solution is then vigorously stirred for a sufficient time to solubilize and evenly distribute all ingredients. During this time a sufficient overlay of inert gas (e.g., N₂) is maintained to substantially prevent introduction of molecular oxygen into the system. Preferably the system is kept protected from sunlight, such as in an ambered vessel, and from other lights emitting ultraviolet light to inhibit photooxidation. The vessel also preferably excludes any metallic materials to minimize contamination with trace metals. The formulation is compounded aseptically under sterile conditions using ingredients suitable for their intended use. Next the solution is taken to ninety percent of its final volume and carefully adjusted to pH 6.5 with sterile 1.0 M HCl and/or 1.0 M NaOH solutions. The solution is then brought to ninety-five percent of its final volume and the isotonicity is adjusted to 290±20 mOsmolar using a sterile saturated sodium chloride solution or neat sodium chloride. The solution is then taken to its final volume with WFI and mixed thoroughly.

### Example 4

### Effect of pH on Thiol Agent Reducing Potential

As noted above, according to non-limiting theory the present invention is believed beneficially to protect quinol compounds from degradation such as oxidative degradation at pH values substantially higher than those previously believed to be capable of accommodating a stabilized quinol composition. This Example is provided by way of illustration and not limitation to show such a beneficial protective effect, as is afforded by inclusion in the present invention compositions and methods of an appropriately selected thiol agent, by virtue of the enhancement of protective reduction potential of the thiol agent as a function of relatively elevated pH. Further according to non-limiting theory, the effect is believed to result from the relationship between (i) the Henderson-Hasselbalch equation as relates to equilibrium distributions of protonated and non-protonated forms of a dissociable acid, and (ii) the Nernst equation as relates to reduction potential of a redox couple.

According to the Henderson-Hasselbalch equation:

pH=pKa+log([A⁻]/[HA]) (I)

The Nernst equation for determining redox potential (E) is classically stated as:

E=E°−(RT/nF)In([C]^{c}[D]^{d}/[A]^{a}[B]^{b}) (II)

At 25° C. and for the following system A+B [symbol] C+D:

Cystine+2H⁺+2e− [symbol] 2Cysteine, (III)

the Nernst equation can be expressed as:

E=E°−(0.05915/n)In([Cysteine]²/[Cystine]¹[H+]²) (IV)

To calculate the reduction potential as a function of pH, the Henderson-Hasselbalch equation is first rearranged to:

[A−]=[HA]×10^{(pH-pKa)} (V)

The fraction X, representing the ratio of protonated to the total [protonated-plus-nonprotonated] forms of cysteine, is next determined, as described by the following:

X=[HA]/([HA]+[A⁻]) (VI)

This equation (VI) can be substituted for [A⁻] from (V), to arrive at:

X=[HA]/([HA]+[HA]×10^{(pH-pKa)}) (VII)

This equation (VII) for X can then be factored to:

X=(1/(1+10^{(pH-pKa)})) (VIII)

To calculate the reduction potential of the cystine/cysteine system as a function of pH, the actual concentration of cysteine (protonated form) is substituted into the Nernst equation to give the following:

E=E°−(0.05915/n)In({[Cysteine](1/(1+10^{(pH-pKa)}))}²/[Cystine]¹[H⁺]²) (IX)

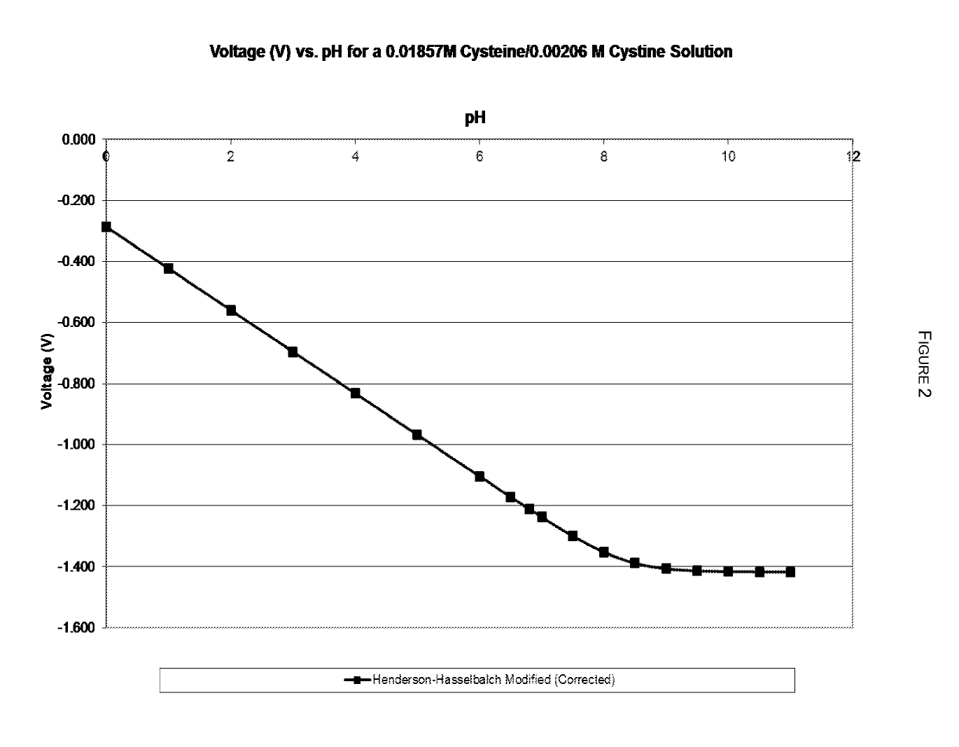

Using the standard reduction potential of cysteine, E°=−0.34 V, the pKa of the cysteine thiol group, pKa=8.3, and the number of electrons for the redox couple, n=2e⁻, the reduction potential (IX) for the cystine/cysteine redox couple can be plotted as a function of pH. Such a plot is depicted, for example, in FIG. 2, for a solution of 2.5 mg/ml cysteine (calculated as ninety percent cysteine and ten percent cystine on a molar basis).

### ADDITIONAL PUBLICATIONS CITED

Bonhomme et al., 1990 Ann. Emerg. Med. 19:1242-1244
Chavdarian et al., 1978 J. Med. Chem. 21:548-554
Chemow et al., 1982 Crit. Care Med. 10:409-416.
Christoph et al., 1988 Ann. Emerg. Med. 17:117-120
Dhalla et al., 1989 Mol. Cell. Biochem. 87:85-92
DiFazio et al., 1986 Anesth. Analg. 65:760-764.
Graham, 1978 Molec. Pharmacol. 14:633-643.
Graham, 1978 Molec. Pharmacol. 14:644-653.
Gorny et al., 2002 Postharvest Biology Technol. 24:271-278.
Hawley et al., 1967 JACS 89:447-450
Holland, 1974 Ann. Ophthalmol. 6:875-888
Hughes et al., 1978 J. Pharm. Pharmac. 30:124-126
Kalyanaraman et al., 1984 J. Biol. Chem. 259:7584
Matthews et al., 1985 Adv. Myocardiol. 6:367-381
Matthews et al., 1985 J. Mol. Cell.Cardiol. 17:339-348
Miram, 1971 Pharm. Prax. 4:89-91
Napolitano et al., 1999 Chem. Res. Toxicol. 12:1090-1097
Noszal et al., 2000 J. Med. Chem. 43:2176-2182
Sokoloski et al., 1962 J. Pharm. Sci. 51:172-177
Springer et al., 1981 Pharmazie 36:706-708
Townsend et al., 1981 Am. J. Hosp. Pharm. 38:1319-1322
Tse et al., 1976 J. Med. Chem. 19:37-40.
Waterman et al., 2002 Pharmaceut. Dev. Technol. 7:1-32
West et al., 1962 J. Pharm. Pharmacol. 14(Suppl):93T-94T
Wollman et al., 1982 Pharmazie 38:37-42.
Church et al., 1994 Am Jl Emerg Med 12(3): 306-309
Murakami et al, 1994 J Dermatol Surg Oncol 20:192-195
Hinshaw et al, 1995 Ophthalmic Sugery 26(3): 194-199
Riemersma et al, 2004 Contact Dermatitis 51: 148
Grubstein et al, 1992 Drug Development and Industrial Pharmacy 18(14): 1549-1566
Stepensky et al, 2003 Jl Pharmaceut Sci 93(4): 969-980
Kirchhoefer et al, 1986 Am Jl Hosp Pharm 43: 1741-1746
Dalton-Bunnow et al, 1985 Am Jl Hosp Pharm 42: 2220-2226
Kalyanaraman et al, 1984 J. Biol. Chem. 259: 354-358
Kirchhoefer et al, 1986 Am Jl Hosp Pharm 43: 1736-1740
Newton et al, 1981 Am Jl Hosp Pharm 38: 1314-1319
Robinson et al, 2000 Anaesthesia 55: 853-858
Smolinske et al, 1992 Clin Toxicol 30(4): 597-606
Campbell et al, 2001 Anesth Prog 48: 21-26
Tse et al, 1976 Jl Med Chem 19(1): 37-40
Hajratwala et al, 1975 Jl Pharmaceut Sci 64(1): 45-48
Schroeter et al, 1958 Jl Am Pharmaceut Assoc 47: 723-728

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A stable pharmaceutical formulation, comprising:
(a) a first composition that comprises at least one quinol compound having a first desired pharmacological activity;
(b) a second composition that comprises at least one local anesthetic compound, said local anesthetic compound comprising at least one amine group that is capable of being reversibly protonated, and being capable of reversibly binding to a voltage-gated Na⁺ channel in a cell membrane to thereby after Na⁺ movement through the voltage-gated Na⁺ channel;
(c) at least one thiol agent; and
(d) at least one pH buffer that maintains a substantially constant pH in the pharmaceutical formulation, wherein the pH is greater than about pH 5.5.

2. A stable pharmaceutical formulation according to claim 1 wherein the quinol compound is present in a reduced form.

3. A stable pharmaceutical formulation according to claim 1 wherein the quinol compound comprises an ortho-quinol moiety or a para-quinol moiety.

4. A stable pharmaceutical formulation according to claim 1 wherein at least one quinol compound comprises a catecholamine.

5. A stable pharmaceutical formulation according to claim 1 wherein at least one quinol compound comprises a compound of Formula (I): 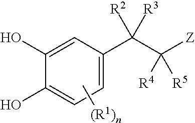
wherein:
n is 0, 1, 2 or 3
each R¹ is the same or different and independently hydrogen, alkyl, hydroxyl, alkoxide, —OC(O)alkyl, —OC(O)aralkyl, aralkyl, amino or halo;
R² and R³ are the same or different and independently hydrogen, hydroxyl, alkoxide, alkyl, oxo, —OC(O)alkyl, —OC(O)aralkyl, amino, monoalkylamino, dialkylamino or halo;
R⁴ and R⁵ are the same or different and independently hydrogen, hydroxyl, alkoxide, —NR⁶₂, —NHNH₂ or lower alkyl,
Z is —NR⁶₂, —COOH or —CR⁷₃;
each R⁶ is the same or different and independently hydrogen, alkyl, aralkyl; or
R⁵ and R⁶ together with the atoms to which they are attached form a heterocycle; and
each R⁷ is the same or different and independently hydrogen, alkyl, aralkyl, —COOH, amino, —C(O)Oalkyl, —C(O)Oaryl, —C(O)Oaralkyl, —NHNH₂, monoalkylamino, dialkylamino,
as a single stereoisomer, a mixture of stereoisomers, or as a racemic mixture of stereoisomers; or as a pharmaceutically acceptable salt thereof.

6. A stable pharmaceutical formulation according to claim 1 wherein the quinol compound comprises a compound selected from the group consisting of 1,2-dihydroxybenzene (catechol, pyrocatechol), 1,4-dihydroxybenzene, epinephrine, norepinephrine, dopamine, dobutamine, isoproterenol, racepinephrine, arbutamine, carbidopa, deoxyepinephrine, dioxethedrine, 3-(3,4-dihydroxyphenyl)-alanine (L-, D- or DL-DOPA), dopexamine, droxidopa, ethylnorepinephrine, hexoprenaline, isoetharine, methyldopa, N-methylepinephrine, nordefrin, rimiterol, epinephrine bitartrate, L-epinephrine-D-hydrogentartate, adrenalone (CAS 99-45-6), arbutamine (CAS 128470-16-6), benserazide (CAS 322-35-0), carbidopa (CAS 28860-95-9), deoxyepinephrine (CAS 501-15-5), dioxethdrine (CAS 497-75-6), dobutamine (CAS 34368-04-02), dopa (CAS 63-84-3), dopamine (CAS 51-61-6), dopexamine (CAS 86197-47-9), droxidopa (CAS 23651-95-8), epinephrine (CAS 51-43-4), ethylnorepinephrine (CAS 536-24-3), fluorodopa (CAS 92812-82-3), hexoprenaline (CAS 3215-70-1), isoetharine (CAS 530-08-5), isoproterenol (CAS 7683-59-2), levodopa (CAS 59-92-7), methyldopa (CAS 555-30-6), N-methylepinephrine (CAS 554-99-4), nordefrin (CAS 6539-57-7), norepinephrine (CAS 51-41-2), protokylol (CAS 136-70-9), rimiterol (CAS 32953-89-2), nordihydroguaiaretic acid and tetrahydropapaveroline (CAS 4747-99-3).

7. A stable pharmaceutical formulation according to claim 1 wherein the quinol compound comprises epinephrine.

8. A stable pharmaceutical formulation according to claim 1 wherein the thiol agent is selected from the group consisting of cysteine, N-acetylcysteine, glutathione, monothioglycerol, cysteine ethyl ester, homocysteine, Coenzyme A, dithiothreitol, 2-mercaptoethanol, 2,3-dimercapto-1-propanol, 2,3-butanedithiol, 2-mercaptoethylamine, ethanedithiol, propanedithiol, 3-mercapto-2-butanol, dimercapto-propane-1-sulfonic acid, dimercaptosuccinic acid, trithiocyanuric acid, 2,5-dimercapto-1,3,4-thiadiazole, 3,4-dimercaptotoluene, 1,4-dimercapto-2,3-butanediol, 1,3-propanedithiol, 1,4-butanedithiol, N-acetylpenicillamine, ACV, N-amyl mercaptan, bucillamine, N-butyl mercaptan, sec-butyl bercaptan, tert-butyl mercaptan, captopril, cysteamine, DBHBT, 2,3-dimercapto-1-propanesulfonic acid, dimercaprol, dithiosalicylic acid, 1,2-ethanedithiol, ethanethiol, isobutyl mercaptan, mecysteine, 2-mercaptoethanol, MESNA, methanethiol, pantetheine, penicillamine, 1,3-propanedithiol, succimer, thioacetic acid, thiobenzyl alcohol, thiocyanic acid, thioglycerol, thioglycolic acid, thiolactic acid, thiomalic acid, thionalide, 1-thiosorbitol, tiopronin, tixocortol and trithiocarbonic acid.

9. A stable pharmaceutical formulation according to claim 1 wherein the thiol agent is selected from the group consisting of cysteine, N-acetylcysteine, glutathione, monothioglycerol, and cysteine ethyl ester.

10. A stable pharmaceutical formulation according to claim 1 wherein the thiol agent is selected from the group consisting of glutathione, monothioglycerol, and cysteine ethyl ester.

11. A stable pharmaceutical formulation according to claim 1 wherein the thiol agent is selected from the group consisting of cysteine and N-acetylcysteine.

12. A stable pharmaceutical formulation according to claim 1 wherein the pH buffer is present under conditions and in sufficient quantity to maintain a pH that is from about pH 5.5 to about pH 9.0.

13. A stable pharmaceutical formulation according to claim 1 wherein the pH buffer is present under conditions and in sufficient quantity to maintain a pH that is from about pH 5.5 to about pH 8.5.

14. A stable pharmaceutical formulation according to claim 1 wherein the pH buffer is present under conditions and in sufficient quantity to maintain a pH that is from about pH 5.5 to about pH 8.25.

15. A stable pharmaceutical formulation according to claim 1 wherein the pH buffer is present under conditions and in sufficient quantity to maintain a pH that is from about pH 5.75 to about pH 7.75.

16. A stable pharmaceutical formulation according to claim 1 wherein the pH buffer is present under conditions and in sufficient quantity to maintain a pH that is from about pH 6.0 to about pH 7.5.

17. A stable pharmaceutical formulation according to claim 1 wherein the pH buffer is present under conditions and in sufficient quantity to maintain a pH that is from about pH 6.3 to about pH 7.3.

18. A stable pharmaceutical formulation according to claim 1 wherein the pH buffer is present under conditions and in sufficient quantity to maintain a pH that is from about pH 6.5 to about pH 7.1.

19. A stable pharmaceutical formulation according to claim 1 wherein the pH buffer is present under conditions and in sufficient quantity to maintain a pH that is from about pH 6.3 to about pH 6.9.

20. A stable pharmaceutical formulation according to claim 1 wherein the pH buffer comprises a compound that is selected from the group consisting of Tris (8.3), Tricine (8.15), citrate (pKa₃=5.4), acetate (4.75), phosphate (7.2), borate (9.24), HEPES (7.55), HEPPS (8), MES (6.15), ACES (6.9), imidazole (7), diethylmalonic acid (7.2), MOPS (7.2), PIPES (6.8), TES (7.5), carbonate, bicarbonate, malate, pyridine, piperazine, succinate, histidine, maleate, Bis-Tris, pyrophosphate, histidine, MOPSO, BES, DIPSO, MOBS, TAPSO, triethanolamine, POPSO, cacodylic acid, ADA, Bis-Tris propane and HEPPSO.

21. A stable pharmaceutical formulation according to claim 1 wherein the pH buffer comprises sodium phosphate.

22. A stable pharmaceutical formulation according to claim 1 wherein the quinol compound comprises epinephrine, the thiol agent is N-acetylcysteine and the pH buffer comprises sodium phosphate.

23. A stable pharmaceutical formulation according to claim 1 wherein the quinol compound comprises epinephrine, the thiol agent is cysteine and the pH buffer comprises sodium phosphate.

24. A stable pharmaceutical formulation according to claim 1 wherein the amine group that is capable of being reversibly protonated has a pKa of from about pH 7.5 to about pH 9.3.

25. A stable pharmaceutical formulation according to claim 1 wherein the amine group that is capable of being reversibly protonated has a pKa of from about pH 7.6 to about pH 9.2.

26. A stable pharmaceutical formulation according to claim 1 wherein the amine group that is capable of being reversibly protonated has a pKa of from about pH 7.7 to about pH 9.1.

27. A stable pharmaceutical formulation according to claim 1 wherein the amine group that is capable of being reversibly protonated has a pKa of from about pH 7.8 to about pH 9.0.

28. A stable pharmaceutical formulation according to claim 1 wherein the amine group that is capable of being reversibly protonated has a pKa of from about pH 7.9 to about pH 8.9.

29. A stable pharmaceutical formulation according to claim 1 wherein the amine group that is capable of being reversibly protonated has a pKa of from about pH 8.0 to about pH 8.8.

30. A stable pharmaceutical formulation according to claim 1 wherein the amine group that is capable of being reversibly protonated has a pKa of from about pH 8.1 to about pH 8.7.

31. A stable pharmaceutical formulation according to claim 1 wherein the amine group that is capable of being reversibly protonated has a pKa of from about pH 8.2 to about pH 8.6.

32. A stable pharmaceutical formulation according to claim 1 wherein the amine group that is capable of being reversibly protonated has a pKa of from about pH 8.3 to about pH 8.5.

33. A stable pharmaceutical formulation according to claim 1 wherein the local anesthetic compound is selected from the group consisting of an amino ester anesthetic and an amino amide anesthetic.

34. A stable pharmaceutical formulation according to claim 1 wherein the cell membrane is a plasma membrane of a neuron.

35. The pharmaceutical formulation of claim 1 wherein the cell membrane is selected from the group consisting of a plasma membrane, a mitochondrial membrane, an endoplasmic reticulum membrane, a lysozomal membrane, an exocytic vacuolar membrane and an endocytic vacuolar membrane.

36. A stable pharmaceutical formulation according to claim 1 wherein the second composition comprises a compound that is selected from the group consisting of lidocaine, propoxycaine, procaine, prilocalne, bupivacaine, Articaine, Benzocaine, Chloroprocaine, Cocaine, Dibucaine, Etidocaine, Hexylcaine, Mepivicaine, Piperocaine, Ropivacaine and Tetracaine.

37. A stable pharmaceutical formulation according to claim 1 wherein the second composition comprises a compound that is selected from the group consisting of lidocaine, bupivacaine, ropivacaine, Articaine, mepivicaine, and prilocalne.

38. A stable pharmaceutical formulation according to claim 1 wherein the first composition comprises epinephrine and the second composition comprises lidocaine.

39. A stable pharmaceutical formulation according to claim 1 wherein the first composition comprises epinephrine, the second composition comprises lidocaine, and the thiol agent comprises N-acetylcysteine.

40. A stable pharmaceutical formulation according to claim 1 wherein the first composition comprises epinephrine, the second composition comprises lidocaine, and the thiol agent comprises cysteine.

41. A stable pharmaceutical formulation according to claim 1 wherein the first composition comprises epinephrine, the second composition comprises lidocaine, the thiol agent comprises N-acetylcysteine and the pH buffer comprises sodium phosphate.

42. A stable pharmaceutical formulation, comprising:
(a) a first composition that comprises at least one quinol compound having a first desired pharmacological activity;
(b) a second composition that comprises at least one local anesthetic compound that is capable of reversibly binding to a voltage-gated Na⁺ channel in a cell membrane to thereby after Na⁺ movement through the voltage-gated Na⁺ channel;
(c) at least one thiol agent; and
(d) at least one pH buffer that maintains a substantially constant pH in the pharmaceutical formulation, wherein the pH is greater than about pH 5.5.

43. The stable pharmaceutical formulation of claim 42 wherein the local anesthetic compound is lidocaine.

44. The stable pharmaceutical formulation of claim 42 wherein the local anesthetic compound is selected from the group consisting of lidocaine, bupivacaine, ropivacaine, Articaine, mepivicaine, and prilocalne.

45. A stable pharmaceutical formulation according to claim 1 wherein the first composition is present at a mass-to-volume ratio in the formulation that is between 1:500,000 and 1:10,000.

46. A stable pharmaceutical formulation according to claim 1 wherein the first composition is present at a mass-to-volume ratio in the formulation that is between 1:250,000 and 1:50,000.

47. A stable pharmaceutical formulation according to claim 1 wherein the thiol agent is present at least from about 0.01 percent weight-to-volume to about ten percent weight-to-volume.

48. A stable pharmaceutical formulation according to claim 1 wherein the thiol agent is present at least from about 0.01 percent weight-to-volume to about one percent weight-to-volume.

49. A stable pharmaceutical formulation according to claim 1 wherein the thiol agent is present at least from about 0.05 percent weight-to-volume to about one percent weight-to-volume.

50. A stable pharmaceutical formulation according to claim 1 wherein the thiol agent is present at least from about 0.1 percent weight-to-volume to about five percent weight-to-volume.

51. A method of stabilizing a pharmaceutical formulation, comprising:
contacting (a) a pharmaceutical formulation,
(b) at least one thiol agent, and
(c) a pH buffer that maintains a substantially constant pH, wherein the pH is greater than about pH 5.5,
wherein:
the pharmaceutical formulation of (a) comprises (i) a first composition that comprises at least one quinol compound having a first desired pharmacological activity, and (ii) a second composition that comprises at least one local anesthetic compound, said local anesthetic compound comprising an amine-containing compound having a second desired pharmacological activity and at least one amine group that is capable of being reversibly protonated, and thereby stabilizing the pharmaceutical formulation.

52. A method of stabilizing a pharmaceutical formulation, comprising:
contacting (a) a pharmaceutical formulation which comprises (i) a first composition that comprises at least one quinol compound having a first desired pharmacological activity, and (ii) a second composition that comprises at least one local anesthetic compound, said local anesthetic compound comprising an amine-containing compound having a second desired pharmacological activity and at least one amine group that is capable of being reversibly protonated,
(b) at least one thiol agent, and
(c) a pH buffer that maintains a substantially constant pH, to produce a stable pharmaceutical formulation.

53. A method of treating a subject, comprising administering to said subject a stable pharmaceutical formulation, comprising:
(a) a first composition that comprises at least one quinol compound having a first desired pharmacological activity;
(b) a second composition that comprises at least one local anesthetic compound, said local anesthetic compound comprising an amine-containing compound having a second desired pharmacological activity and at least one amine group that is capable of being reversibly protonated;
(c) at least one thiol agent; and
(d) at least one pH buffer that maintains a substantially constant pH in the pharmaceutical formulation, wherein the pH is greater than about pH 5.5.

54. A method for the manufacture of a medicament for therapeutic treatment of a subject with a stable pharmaceutical formulation, said method comprising:
contacting (a) a pharmaceutical formulation,
(b) at least one thiol agent, and
(c) a pH buffer that maintains a substantially constant pH, wherein the pH is greater than about pH 5.5,
wherein:
the pharmaceutical formulation of (a) comprises (i) a first composition that comprises at least one quinol compound having a first desired pharmacological activity, and (ii) a second composition that comprises at least one local anesthetic compound, said local anesthetic compound comprising an amine-containing compound having a second desired pharmacological activity and at least one amine group that is capable of being reversibly protonated.
